(19) United States
(12) Patent Application Publication
(10) Pub. No.: US 2020/0289625 A1
(43) Pub. Date: Sep. 17, 2020

(54) Uses of Irisin

(71) Applicant: TEXAS A&M UNIVERSITY, College Station, TX (US)

(72) Inventors: Corinne Metzger, Indianapolis, IN (US); Anand Narayanan, Tallahassee, FL (US); David Zawieja, Temple, TX (US); Susan Bloomfield, College Station, TX (US)

(73) Assignee: Texas A&M University System, College Station, TX (US)

(21) Appl. No.: 16/759,644

(22) PCT Filed: Oct. 25, 2018
(86) PCT No.: PCT/US2018/057508

§ 371 Date: Apr. 27, 2020

Related U.S. Application Data
(60) Provisional application No. 62/577,036, filed on Oct. 25, 2017.

(51) Int. Cl. A61K 38/39; A61P 19/08

(52) U.S. Cl. CPC A61K 38/39; A61P 19/08

(57) ABSTRACT

The present invention provides a method for treating inflammatory conditions, for example, inflammatory bowel disease, ulcerative colitis induced inflammatory bowel disease, spinal cord injury or spaceflight-induced immune dysregulation and associated comorbidities in a mammal or subject in need of such treatment. The present invention also provides a method for decreasing osteocyte protein levels in a mammal suffering from inflammatory condition. A pharmacologically effective does of Irisin or a pharmaceutical composition of irisin is administered to the mammal or subject one or more times.

### CROSS-REFERENCE TO RELATED APPLICATIONS

This international patent application claims benefit of priority under 35 U.S.C. § 119(e) of provisional application U.S. Ser. No. 62/577,036, filed Oct. 25, 2017, the entirety of which is hereby incorporated by reference.

### GOVERNMENT SUPPORT CLAUSE

This invention was made with government support under grant number U01HL123420 awarded by the National Institutes of Health. The government has certain rights in the invention.

### BACKGROUND OF THE INVENTION

### Field of the Invention

The present invention relates generally to treatment of chronic inflammatory conditions and diseases. More specifically, the present invention relates to treatment of inflammatory disorders using irisin.

### Detailed Description of the Invention

The lymphatics are the main transport path of fluid and elements from the parenchymal tissues to the lymph nodes via the afferent lymphatics, and from the nodes to systemic circulation via the efferent lymphatics. A functional lymphatic vascular network is necessary for delivery of lymph contents representing the immunological state of the drained parenchyma to the lymph node to allow for appropriate immunological responses. Inflammation-induced lymphangiogenesis, or the formation of new lymphatic structures, occurs in pathologies such as inflammatory bowel disease (IBD), where the intestinal lymphatics proliferate from their normal topology in the submucosa to every layer of the inflamed small and large bowel. It is not fully understood what drives this uncontrolled lymphatic infiltration, if it affects function or how local cytokines (known to be significantly altered in inflammatory bowel disease patients) associate with GI lymphatic changes.

Inflammatory bowel disease causes comorbidities including osteoporosis and elevated fracture risk. This inflammation-induced bone loss is characterized by increased osteocyte expression of the proteins receptor activator of nuclear factor κB ligand (RANKL), TNF-α, and IL-6, increased osteoclasts, and decreased bone formation. It is unknown, however, whether the immunological processes in inflammatory bowel disease that are driving bone loss are distinct or parallel to those in the gut. Furthermore, all current treatments for inflammatory bowel disease aim to mitigate disease symptoms, but have significant negative consequences. Anti-cytokine treatments, like anti-TNF, increase infection risks. Additionally, anti-TNF treatments can cause autoantibody development and the onset of other autoimmune diseases. Corticosteroids further exacerbate bone loss as well as cause detrimental metabolic changes. Therefore, the development of treatments to effectively mitigate inflammation in the gut and extra-intestinal sites are needed for inflammatory bowel disease patients.

Exercise induces peroxisome proliferator-activated receptor-gamma coactivator 1 alpha (PGC-1α) in muscle, which in turn increases the expression and secretion of the adipomyokine irisin. Irisin has recently emerged as a potential modulator of exercise-related systemic physiological adaptations. Muscle specific knockout of PGC-1α results in local muscle inflammatory gene upregulation. Some studies have shown correlations of lower serum irisin with pathologies that have some element of inflammation Additionally, recent studies have demonstrated that exogenous irisin treatment improved tissue functions in lung, brain, and vascular endothelium in various rodent pathological models that again have some element of inflammation. Irisin has also recently emerged as a bone anabolic factor.

Thus, there is a recognized need in the art for additional therapies for treating inflammatory conditions. Particularly, the art is deficient in treating chronic, systemic inflammatory conditions with irisin. The present invention fulfils this long-standing need and desire in the art.

### SUMMARY OF THE INVENTION

The present invention is directed to a method for treating an inflammatory condition in a subject in need of such treatment. The method comprises the step of administering one or more times to the subject a pharmacologically effective dose of irisin or a pharmaceutical composition thereof.

The present invention also is directed to a related method for treating an inflammatory bowel disease in a subject. The method comprises administering a pharmacologically effective dose of irisin or a pharmaceutical composition thereof one or more times to the subject.

The present invention is directed further to a related method for treating spinal cord injury in a subject. The method comprises administering a pharmacologically effective dose of irisin or a pharmaceutical composition thereof one or more times to the subject.

The present invention is directed further to a related method for treating spaceflight-induced immune dysregulation in a subject. The method comprises administering a pharmacologically effective dose of irisin or a pharmaceutical composition thereof one or more times to the subject.

The present invention is directed further still to a method for decreasing a level of an osteocyte protein in a mammal suffering from an inflammatory condition. The method comprises administering one or more times to the mammal a pharmacologically effective dose of irisin or a pharmaceutical composition thereof.

Other and further aspects, features, and advantages of the present invention will be apparent from the following description of the presently preferred embodiments of the invention given for the purpose of disclosure.

### BRIEF DESCRIPTION OF THE DRAWINGS

Notwithstanding any other forms which may fall within the scope of the present invention, a preferred embodiment/preferred embodiments of the invention will now be described, by way of example only, with reference to the accompanying drawings in which:

FIGS. 1A-1B show body weights and colon histopathology in response to TNBS-induced inflammatory bowel disease in a rodent model. FIG. 1A: Body weights were no different in any group throughout the entire study. FIG. 1B: Aggregated colon histopathology scores validate gut inflammation and damage in the TNBS rats. TNBS+Ir was no different from the two Veh treated groups. *Different from all other groups (p<0.05). All error bars shown are for standard deviation.

FIGS. 2A-2E show podoplanin, a marker of lymphatic vessels, immunofluorescence in the distal colon as part of the TNBS-induced inflammatory bowel disease study. FIG. 2A: Representative images of podoplanin for each group in the distal colon at 20× magnification. FIG. 2B: Podoplanin integrated density (integrated density, ID=mean fluorescence intensity*region of interest area) was higher in TNBS mucosa compared to all other groups. FIG. 2C: Podoplanin integrated density was higher in the TNBS submucosal compartment compared to all other groups. FIG. 2D: There were no statistical differences in the maximum diameter of podoplanin-positive vessels in the submucosa, but a modest, but not statistically different, elevation in the TNBS groups. FIG. 2E: The minimum diameter of podoplanin-positive vessels was greater in TNBS compared to all other groups. All error bars shown are for standard deviation. *Different from all other groups (p<0.05). Box in top right shows p-values for main effect of TNBS, Irisin treatment (Ir), and TNBS-by-Irisin interaction (TNBS*Ir).

FIGS. 3A-3E depict experiments relating to Gut TNF-α as part of the TNBS-induced inflammatory bowel disease study. FIG. 3A: Representative images of distal colon TNF-α (top row) and TNF-α +podoplanin overlay (bottom row). FIG. 3B: Integrated density of TNF-α was higher in the TNBS mucosa compared to all other groups. FIG. 3C: Integrated density of TNF-α was elevated in the TNBS submucosal area compared to all other groups. FIG. 3D: The number of TNF-a+cells was highest in TNBS followed by both Ir groups with Veh having the lowest number. FIG. 3E: In the submucosa, the number of TNF-α+ cells was elevated in TNBS compared to all other groups. *Different from all other groups (p<0.05). Bars not sharing the same letter are different. Box in top right shows p-values for main effect of TNBS, Irisin treatment (Ir), and TNBS-by-Irisin interaction (TNBS*Ir). Error bars shown are for standard deviation.

FIGS. 4A-4C depict experiments relating to Gut RANKL as part of the TNBS-induced inflammatory bowel disease study. FIG. 4A: Representative images of distal colon RANKL (top row) and RANKL+podoplanin overlay (bottom row). FIG. 4B: Integrated density of RANKL was higher in the TNBS mucosa compared to all other groups. FIG. 4C: Integrated density of RANKL was elevated in the TNBS submucosal area compared to all other groups. *Different from all other groups (p<0.05). Box in top right shows p-values for main effect of TNBS, Irisin treatment (Ir), and TNBS-by-Irisin interaction (TNBS*Ir). Error bars shown are for standard deviation.

FIGS. 5A-5F show immunofluorescence results of IL-10, IL-4, and IFN-γ in the distal colon as part of the TNBS-induced inflammatory bowel disease study. FIG. 5A: Integrated density of IL-10 was slightly decreased due to irisin treatment with TNBS statistically higher than Veh+Ir. FIG. 5B: Integrated density of IL-4 was not statistically different across the four groups, but a main effect of TNBS was observed. FIG. 5C: Integrated density of IFN-γ was elevated due to irisin treatment. FIG. 5D: Representative images of IL-10. FIG. 5E:

Representative images of IL-4. FIG. 5F: Representative images of IFN-γ. *Different from all other groups (p<0.05). Bars not sharing the same letter are different. Box in top right shows p-values for main effect of TNBS, Irisin treatment (Ir), and TNBS-by-Irisin interaction (TNBS*Ir). Error bars shown are for standard deviation.

FIGS. 6A-6F show bone histomorphometry as part of the TNBS-induced inflammatory bowel disease study. FIG. 6A: Cancellous bone formation rate at the proximal tibia and 4^{th} lumbar vertebrae were depressed due to TNBS and elevated due to irisin-treatment. FIG. 6B: Representative image of single versus double label fluorochrome in cancellous bone. FIG. 6C: Cancellous osteoid surface in TNBS was lower than all other groups in both the proximal tibia and L4. FIG. 6D: Representative image of osteoid surface. FIG. 6E: Osteoclast surface was increased in TNBS, but decreased due to irisin. FIG. 6F: Representative image of osteoclast surface. *Different from all other groups (p<0.05). Bars not sharing the same letter are statistically different. Box in top right shows p-values for main effect of TNBS, Irisin treatment (Ir), and TNBS-by-Irisin interaction (TNBS*Ir). Error bars shown are for standard deviation.

FIGS. 7A-7H show experiments with Osteocyte proteins as part of the TNBS-induced IBD study. FIG. 7A: TNF-α-positive osteocytes were higher in TNBS and lower due to irisin. FIG. 7B: IL-6-positive osteocytes were higher in TNBS, but levels were lowered due to irisin. FIG. 7C: TNBS had the highest sclerostin positive osteocytes with irisin decreasing sclerostin. FIG. 7D: Annexin V-positive osteocytes (a measure of apoptosis) was higher in TNBS than all other groups. FIG. 7E: RANKL-positive osteocytes were highest in TNBS. FIG. 7F: OPG-positive osteocytes were highest in TNBS. FIG. 7G: IL-10-positive osteocytes were lower in irisin-treated animals. FIG. 7H: IL-4 positive osteocytes were lower in irisin-treated animals. *Different from all other groups (p<0.05). Bars not sharing the same letter are statistically different. Box in top right shows p-values for main effect of TNBS, Irisin treatment (Ir), and TNBS-by-Irisin interaction (TNBS*Ir). Error bars shown are for standard deviation.

FIGS. 8A-8B show colon histopathology and immunofluorescence labeling as part of the DSS-induced inflammatory bowel disease study in a rodent model. FIG. 8A shows aggregated colon histopathology scores validate gut inflammation and damage in the (dextran sulfate sodium) DSS rats. DSS+Ir had significantly lower gut inflammation with the DSS group, but was not the same as the two control groups. FIG. 8B shows distal colon immunofluorescence quantification of TNF-α, a canonical marker for pro-inflammation. DSS had elevated mucosa and statistically elevated submucosa TNF-α levels, with irisin treatment returning DSS+Ir TNF-α quantity in both mucosa and submucosa compartments to control levels.

FIGS. 9A-9B show bone histomorphometry as part of the DSS-induced IBD study. FIG. 9A shows that cancellous bone formation rate at the proximal tibia were depressed due to DSS and elevated due to irisin-treatment. FIG. 9B. shows that osteoclast surface was increased in DSS, but decreased due to irisin.

FIGS. 10A-10C show experiments with osteocyte proteins as part of the DSS-induced IBD study. FIG. 10A shows TNF-a-positive osteocytes were higher in DSS and lower due to irisin. FIG. 10B shows RANKL-positive osteocytes were highest in DSS, but decreased after treatment with Irisin. FIG. 10C shows that DSS had the highest sclerostin positive osteocytes with irisin decreasing sclerostin.

FIGS. 11A-11B show bone histomorphometry as part of the spinal cord injury (SCI) study in a rodent model. FIG. 11A shows that cancellous bone formation rate at the proximal tibia were depressed due to spinal cord injury and elevated due to irisin-treatment. FIG. 11B shows that osteoclast surface was increased in spinal cord injury, but decreased due to irisin.

FIGS. 12A-12C show experiments with osteocyte proteins as part of the SCI-study. FIG. 12A shows TNF-α-positive osteocytes were higher in spinal cord injury and lower due to irisin. FIG. 12B shows RANKL-positive osteocytes were highest in spinal cord injury which decreased after treatment with Irisin. FIG. 12C shows that spinal cord injury had the highest sclerostin positive osteocytes with irisin decreasing sclerostin.

FIGS. 13A-13B show colon histopathology and immunofluorescence labeling as part of the spaceflight-analogue ground-based rodent model, hindlimb unloading (HU). FIG. 13A shows aggregated colon histopathology scores validate gut inflammation and damage in the HU rats. HU+Ir was had significantly lower gut inflammation compared to the two control groups. FIG. 13B shows distal colon immunofluorescence quantification of TNF-α, a canonical marker for pro-inflammation. HU had elevated mucosa TNF-α levels, with irisin treatment returning HU+Ir TNF-α quantity in the mucosa compartment to control levels.

FIGS. 14A-14B show bone histomorphometry as part of the spaceflight-analogue HU study. FIG. 14A shows that cancellous bone formation rate at the proximal tibia were depressed due to hindlimb unloading and elevated due to irisin-treatment. FIG. 14B shows that osteoclast surface was increased in hindlimb unloading, but decreased due to irisin. FIGS. 15A-15C show experiments with osteocyte proteins as part of the spaceflight-analogue HU study. FIG. 15A shows TNF-α-positive osteocytes were higher in hindlimb unloading and lower due to irisin. FIG. 15B shows RANKL-positive osteocytes were highest in hindlimb unloading which decreased after treatment with Irisin. FIG. 15C shows that hindlimb unloading had the highest sclerostin positive osteocytes with irisin decreasing sclerostin.

### DETAILED DESCRIPTION OF THE INVENTION

As used herein, “a” or “an” may mean one or more. As used herein in the claim(s), when used in conjunction with the word “comprising”, the words “a” or “an” may mean one or more than one.

As used herein “another” or “other” may mean at least a second or more of the same or different claim element or components thereof. Similarly, the word “or” is intended to include “and” unless the context clearly indicates otherwise.

As used herein, “comprise” or “comprises” or “comprising”, except where the context requires otherwise due to express language or necessary implication, are used in an inclusive sense, i.e. to specify the presence of the stated features but not to preclude the presence or addition of further features in various embodiments of the invention.

As used herein, “including”, “which includes” or “that includes” is also an open term that also means including at least the elements/features that follow the term, but not excluding others. Thus, including is synonymous with and means comprising.

As used herein, the term “about” refers to a numeric value, including, for example, whole numbers, fractions, and percentages, whether or not explicitly indicated. The term “about” generally refers to a range of numerical values (e.g., +/−5-10% of the recited value) that one of ordinary skill in the art would consider equivalent to the recited value (e.g., having the same function or result). In some instances, the term “about” may include numerical values that are rounded to the nearest significant figure.

As used herein, the ordinal adjectives “first”, “second”, “third”, etc., unless otherwise specified are used to describe a common object, merely indicate that different instances of like objects are being referred to, and are not intended to imply that the objects so described must be in a given sequence, either temporally, spatially, in ranking, or in any other manner.

As used herein, the term “systemic” refers generally to the whole body of a subject, for example, a mammal.

As used herein, the term “mammal” or “subject” refers generally to any recipient of at least irisin, for example, but not limited to, a human, a rodent (rat and mouse), and a pig. Mammal is interchangeable with “subject”.

As used herein “pharmacologically effective dose” generally refers to an amount of irisin or a pharmaceutical composition thereof effective to accomplish the intended purpose. However, the amount may be less than that amount when a plurality of doses is to be administered, i.e., the total effective amount can be administered in cumulative dosage units. The amount of irisin also may be more than the effective amount when the composition provides sustained release of the irisin. The total amount of irisin to be used may be determined by methods well known to those of ordinary skill in the art.

In one embodiment of the present invention, there is provided a method for treating an inflammatory condition in a subject in need of such treatment, comprising the step of administering one or more times to the subject a pharmacologically effective dose of irisin or a pharmaceutical composition thereof.

In this embodiment, the pharmacologically effective dose is from about 50 ng/kg of the subject's weight to about 70 ng/kg of the subject's weight. In this embodiment the subject may be a human, a rodent, or a pig. Representative inflammatory conditions include but are not limited to inflammatory bowel disease (including Crohn's disease and ulcerative colitis), microscopic colitis, Behcet's disease, inflammatory bone loss, primary sclerosing cholangitis, uveitis, rheumatoid/psoriatic arthritis, psoriasis, systemic lupus erythematosus, spinal cord injury, traumatic brain injury, lymphedema, or spaceflight-induced immune dysregulation and associated comorbidities.

In one aspect of this embodiment, administering irisin to the subject may decrease TNF-α⁺ cells and expression of TNF-α+. In another aspect of this embodiment, administering irisin to the subject may decrease RANKL, and IL-6 expression therein. In yet another aspect, administering irisin to the subject may increases IFN-γ expression therein. In yet another aspect, administering irisin to the subject may inhibits development of abnormally-structured lymphatic hyperproliferation and may reduce overexpression of podoplanin in lymphatic vessels that is associated in inflammation-induced lymphangiogenesis. In yet another aspect, administering irisin to the subject may inhibit development of secondary/tertiary lymphoid aggregates therein. In yet another aspect, administering irisin to the subject decreases osteoclast surface and increases osteoid surface and bone formation rate to reduce inflammation-induced alterations in bone turnover. In yet another aspect, administering irisin to the subject decreases the level of at least one osteocyte protein. In this aspect, the osteocyte protein may be TNF-α, IL-6, sclerostin, RANKL, OPG or a combination thereof.

In another embodiment of the present invention, there is provided a method for treating an inflammatory bowel disease in a subject in need of such treatment, comprising the step of administering one or more times to the subject a pharmacologically effective dose of irisin or a pharmaceutical composition thereof. In this embodiment, the pharmacologically effective dose is from about 50 ng/kg of the subject's weight to about 70 ng/kg of the subject's weight. Representative examples of an inflammatory bowel disease are Crohn's disease or ulcerative colitis. In this embodiment the subject may be a mammal. Representative examples of a mammal are a human, a rodent, or a pig.

In yet another embodiment of the present invention there is provided a method for treating spinal cord injury in a subject in need of such treatment, comprising the step of administering one or more times to the subject a pharmacologically effective dose of irisin or a pharmaceutical composition thereof.

In this embodiment irisin may be administered to the subject in a dose from about 50 ng/Kg of the subject's weight to about 70 ng/Kg of the subject's weight. Also in this embodiment the subject may be a mammal. Representative examples of a mammal are a human, a rat or a mouse or other rodent, or a pig.

In one aspect of this embodiment administering irisin to the subject decreases osteoclast surface and increases bone formation rate to reduce inflammation-induced alterations in bone turnover.

In one another aspect administering irisin to the subject may result in decreased level of osteocyte proteins therein. In this aspect the osteocyte protein may be TNF-α, sclerostin, or RANKL

In yet another embodiment of the present invention, there is provided a method for treating spaceflight-induced immune dysregulation in a subject in need of such treatment, comprising the step of administering one or more times to the subject a pharmacologically effective dose of irisin or a pharmaceutical composition thereof.

In this embodiment said pharmacologically effective dose is from about 50 ng/kg of the subject's weight to about 70 ng/kg of the subject's weight. In this embodiment the subject may be a mammal. Representative examples of a mammal are a human, a rodent, or a pig. Representative inflammatory conditions include but are not limited to spaceflight-induced immune dysregulation including immune dysregulation and suppression, cardiovascular dysfunction, lymphatic dysfunction, gastrointestinal dysfunction, inflammation-induced bone loss.

In one aspect of this embodiment, administering irisin to the subject may decrease TNF-α⁺ expression therein. In another aspect, administering irisin to the subject decreases osteoclast surface and increases bone formation rate mitigating inflammation-induced alterations in bone turnover. In yet another aspect, administering irisin to the subject decreases a level of at least one osteocyte protein therein. In this aspect, the osteocyte protein may be TNF-α, sclerostin, RANKL or a combination thereof.

In yet another embodiment of the present invention, there is provided a method for decreasing an osteocyte protein level in a mammal suffering from an inflammatory condition comprising the step of administering one or more times to the mammal a pharmacologically effective dose of irisin or a pharmaceutical composition thereof. In this embodiment, the osteocyte protein is TNF-α, sclerostin, RANKL or a combination thereof. Representative examples of the inflammatory condition are inflammatory bowel disease, spinal cord injury, or spaceflight-induced immune dysregulation and associated comorbidities. Representative examples of the mammal are a human, a rodent or a pig.

In this embodiment, the pharmacologically effective dose is from about 50 ng/kg of the mammal's weight to about 70 ng/kg.

In one aspect of this embodiment, administering irisin to the mammal decreases osteoclast surface and increases bone formation rate in the mammal suffering from inflammatory condition to reduce inflammation-induced alterations in bone turnover.

Provided herein are methods for treating chronic, systemic inflammatory conditions with exogenous irisin. Examples of these conditions include, but are not limited to, inflammatory bowel disease (including Crohn's disease, ulcerative colitis, microscopic colitis, Behcet's disease) and their co-morbidities (inflammatory bone loss, primary sclerosing cholangitis, uveitis), other autoimmune diseases (rheumatoid/psoriatic arthritis, psoriasis, systemic lupus erythematosus, etc), as well as conditions such as spinal cord injury, traumatic brain injury, lymphedema, and spaceflight-induced immune dysregulation. The present invention demonstrates that irisin, a factor naturally released from exercising muscle, ameliorated both GI and bone inflammation in chronic inflammatory bowel disease rodent models, as well as restored the GI lymphatic and bone architecture.

A chemically-based inflammatory bowel disease model (TNBS colonic instillation) was employed. Two other randomized age-matched groups of animals were treated with Irisin (Veh+Ir and TNBS+Ir). The GI tissues were assessed via histology, as well as via immunofluorescence, to determine specific local tissue changes in lymphatic structures and immune factors called cytokines. Pertinent parameters at both sites were quantified and compared across all the 4 groups—Vehicle (Veh,) Vehicle with irisin treatment (Veh+Ir), inflammatory bowel disease via TNBS (TNBS), and inflammatory bowel disease with irisin treatment (TNBS+Ir). There was significant pathology in the TNBS group as compared to Veh groups, with significant recovery from pathology in the irisin-treated TNBS animals. No significant side-effects were seen in the control (healthy) animals nor the inflammatory bowel disease animals treated with irisin.

TNBS animals had disrupted intestinal epithelial lining, an increase in cell density in the mucosal area of the colon, as well as evidence for edema based on thickness of the muscle wall and submucosal area. In the TNBS+Ir animals, these characteristics were resolved and the colonic structure was comparable to Vehicle animals. As impairments of the normal colonic structure are associated with inflammation and changes in the lymphatic architecture, the colonic lymphatic topography was characterized using immunofluorescence podoplanin staining. In TNBS animals there is a stark infiltration and increase in density of podoplanin-positive regions in the colonic mucosa compared to Veh animals, as well as a significant increase in podoplanin signal intensity, even when accounting for the increased area of podoplanin-positive regions. TNBS+Ir animals had restored podoplanin architecture, with Veh+Ir being no different from Veh. Various proteins involved in inflammatory signalling, i.e. cytokines including TNF-a, IFN-Y, IL-10, IL-4, that are associated with influencing lymphatic architecture were assessed in the colon as well. Notably TNF-α was significantly elevated in TNBS animals in both number of cells as well as protein expression, but was drastically reduced in TNBS+Ir animals. This is a crucial discovery, as TNF-α is a classical pro-inflammatory cytokine elevated in multiple conditions, including inflammatory bowel disease, but minimally assessed in the local tissue. Similarly to what was observed with TNF-α, Irisin treatment ameliorated the pathological increase in RANKL.

Using a chronic TNBS injection model of inflammatory bowel disease in rats, physical, histological features of chronic colonic inflammation were associated with significant infiltration of podoplanin^{hi} lymphatic structures into the mucosal lamina propria, comparable to what is seen in inflammatory bowel disease patients. Morphologically these podoplanin^{hi} lymphatic regions in TNBS animals do not form typical lymph-capillary networks or pre-collector/collecting vessel structures, based on their lack of defined borders/lumen. The present invention examined how the inflammatory bowel disease pathology in the GI is characterized by specific inflammatory/immunological mechanisms that drove analogous changes in bone. These inflammation-induced changes in bone turnover are characterized by increased osteocyte Th₁ cytokines TNF-α and IL-6, with no change or lowered IL-10 and IL-4, respectively, similar to the response in the colon. These data suggest and support a paradigm in which inflammation in one organ bed can lead to similar adaptations at a distant site, with information from the local originating site being carried via lymph to the draining node whose responses results in systemic effects at distant sites. Furthermore, modulating the local inflammatory changes via exogenous irisin improved lymphatic outcomes at the site of damage leading to a resolution of inflammation in both local/gut and distant sites/bone.

Irisin treatment significantly increased bone formation in both TNBS+Ir and Veh+Ir to levels above the comparable Veh animals suggesting a strong anabolic effect of Irisin on osteoblast function. Osteoclast surface provides an index of how much bone surface area is covered by osteoclasts and presumably undergoing bone resorption; indeed, in TNBS animals, osteoclast surface was elevated. Irisin treatment lowered osteoclast surface in both TNBS and Veh groups, supporting decreased resorption. Therefore, the irisin treatment resulted in increased bone formation and decreased bone resorption, the opposite of what is seen with TNBS. There was a significant increase in osteocytes positive for RANKL, a key factor that stimulates osteoclasts development, in TNBS; Irisin ameliorated this effect. Furthermore, TNF-α is an agonist of RANKL and a pro-inflammatory cytokine that also increases osteoclasts as well as suppresses osteoblasts and was also elevated in TNBS animals but was also reduced in Irisin treated animals. These data suggest a strong anti-inflammatory and anabolic effect of Irisin in bone with irisin treatment reversing the negative inflammatory effect of inflammatory bowel disease on bone.

Thus, irisin treatment mitigates the pathological changes in gut structure and edema due to inflammatory bowel disease. Irisin treatment reorganizes the aberrant local lymphatic architecture in the colon, which in inflammatory bowel disease proliferate and lose their normal morphology. Irisin treatment modulates the inflammatory cytokines in the inflammatory bowel disease colon and bone, reducing the overall magnitude but also shifting the directionality of the immunological response. Irisin reverses the increase in bone resorption and the decrease in bone formation rate observed in inflammatory bowel disease animals. Irisin treatment suppresses the increase in RANKL expression seen in both gut and bone tissue during inflammatory bowel disease.

RANKL downregulation in TNBS+Ir suggests inhibition of the development of these lymphoid aggregates typical in inflammatory bowel disease. The present invention demonstrated elevated mucosal and submucosal RANKL in TNBS animals associated with elevated densities of TNF-α⁺ cells and podoplanin^{hi} lymphatic areas, suggesting a mechanistic role of RANKL in colonic lymphoid aggregate development in inflammatory bowel disease. The present invention demonstrated that exogenous irisin treatment modulated the local and distant inflammatory milieu in inflammatory bowel disease vholistically. There was a complete amelioration of the TNF-α/RANKL driven pathogenesis of colonic lymphatic hyper-proliferation at the originating site of inflammation, as well as a reduction in osteoclast function and increased bone formation at a distant site of inflammation with irisin treatment. Irisin blocked the elevated TNF-α⁺ cell numbers in the gut of TNBS+Ir animals and increased TNF-α⁺ osteocytes. Similar reductions with RANKL were seen in both gut and bone. Concurrent with these changes, irisin treatment restored colonic lymphatic architecture as well by notably reducing lymphoid aggregates. Irisin likely has direct effects on lymphatics, driving the amelioration of gut inflammatory processes and distant inflammatory processes.

The present invention demonstrated lymphatic alterations associated with elevated colon TNF-α and RANKL levels and gut inflammatory damage in chronic TNBS-induced colitis. These changes in the gut were paralleled immunologically in the bone leading to increased bone resorption and decreased bone formation. The present invention demonstrates for the first time that exogenous treatment with irisin blocked the gut inflammatory changes, improved lymphatic structure and bone turnover likely by reducing TNF-α/RANKL. Thus, irisin is a holistic treatment that could mitigate chronic inflammatory conditions.

The following example(s) are given for the purpose of illustrating various embodiments of the invention and are not meant to limit the present invention in any fashion.

### EXAMPLE 1

### Materials and Methods

### Animals

Sprague-Dawley rats (male, 1.5 months old) were ordered from Envigo (Houston, Texas) and singly housed in a facility with 12 hour light dark cycles. Gut inflammation in a rodent model of inflamed bowel disease was induced by rectal instillations of 1 uL/gram body weight, of 2,4,6-trinitrobenzenesulfonic acid (TNBS; 30 mg/kg, Sigma Aldrich, St Louis, Mo.) in 30% ethanol:DiH₂O solutions (on days 1, 7, 14, 21 and 28) as described (Metzger 2017). This model is analogous to Crohn's disease pathology, and the TNBS treatment regimen follows a mild-moderate level of inflammation. At two months of age, animals were randomly divided into four different groups (n=8/group): Vehicle (Veh), Vehicle with irisin (Veh+Ir), IBD (TNBS), and inflammatory bowel disease with irisin (TNBS+Ir). All animal procedures were approved by the Texas A&M Institutional Animal Use and Care Committee and confirm to the NIH Guide for the Care and Use of Laboratory Animals.

### Irisin Injections

Recombinant irisin (Adipogen Life Sciences, San Diego, Calif.) was dissolved in sterile phosphate buffered saline. Doses of 18 ng/mL were injected intraperitoneally into all irisin treated rats. Irisin treated rats received two dose per week (3.5 days apart) for 3 weeks, with the first irisin injection coincided with the second TNBS instillation.

### Tissue Processing and Histological Analysis

Whole length colons were removed, processed, and were scored from H&E stained sections based on: epithelial structure, crypt structure, cellularity, and edema (assessed by separation between the mucosa and muscularis externa layers). Scores were adjusted to account for area of tissue affected. All scores were conducted blindly.

### Colonic Immunofluorescence

Tissues from the colon were collected, flushed and processed for paraffin or OCT embedding. Some tissues were fixed in 4% PFA overnight and embedded in paraffin. The paraffin sections (10-um) were deparaffinized, blocked for 30 m with 2.5% Goat Serum:PBS at room temperature, and incubated overnight at 4° C. with primary antibody combinations of anti-podoplanin (Novus Biologicals), anti-TNF-α (LifeSpan BioSciences, Inc), and anti-RANKL (Abcam). Frozen tissues sections (10-um) were fixed in 4% PFA for 45 minutes at room temperature, and incubated overnight at 4° C. with primary antibody combinations for anti-podoplanin and anti-IL-4 (Abcam), IL-10 (Abcam), IFN-γ (Abcam). Sections were incubated with corresponding secondary antibodies for Mouse IgG1 Alexa Fluor-488 and Rabbit IgG Alexa-Fluor 633 (Fisher Scientific) for 1h at room temperature in the dark. Sections were then mounted in Prolong Gold Antifade with DAPI and imaged by confocal microscopy (Olympus Fluoview 300). Images (1024×1024) were acquired at 20× objective at 5 random fields with z-stack slices of 2 microns. Z-stacks were imported into ImageJ v.1.51 and quantified consistently across groups. (Fluorescence Integrated Density [ID]=region of interest area×the mean fluorescence intensity).

Dynamic and Static Histomorphometry

Undemineralized right proximal tibia and fourth lumbar vertebrae (L4) were fixed in 4% phosphate buffered formalin and then subjected to serial dehydration and embedded in methyl methacrylate (J. T. Baker, VWR, Radnor, Pa.). Serial frontal sections (8 μm thick) were left unstained for analysis of fluorochrome labels (6 days apart in last week) for dynamic histomorphometry with measures including mineralized bone surface (MS/BS), mineral apposition rate (MAR) and bone formation rate (BFR) as described (Metzger 2017). Additional frontal sections of the proximal tibia and L4 (4 μm thick) were treated with a Von Kossa stain+tetrachrome counterstain and imaged at 40× magnification for identification of osteoclast surface (OcS/BS) and osteoid surface (OS/BS) as described (Metzger 2017). All analyses were completed on OsteoMeasure Analysis System, version 3.3 (OsteoMetrics, Inc., Atlanta, Ga.) by the same individual to ensure consistency in all measures. All nomenclature for cancellous histomorphometry follows standard usage (Dempster 2013).

### Immunohistochemistry of Osteocyte Proteins

Left distal femurs were fixed in 4% phosphate buffered formalin and then stored in 70% ethanol prior to decalcification in a formic acid/sodium citrate solution. Tissues were then paraffinized and 8 μm sections were immunostained as described¹⁶ with the following primary antibodies: anti-TNF-α, anti-IL-6 (Abcam), anti-IL-10 (Abcam), anti-IL-4 (Abcam), anti-annexin V (Abcam), anti-sclerostin (R&D Systems, Minneapolis, Minn.), anti-RANKL, and anti-OPG (Biorbyt, San Francisco, Calif.). All sections were counterstained with methyl green. Sections were analyzed as the percentage of osteocytes stained positively for the protein with a 4 mm² region in the distal femur cancellous bone as previously described (Metzger Narayanan 2017). All analyses were completed by the same individual.

### Statistical Analyses

Data were analyzed as a 2×2 factorial design (TNBS by irisin). If the model 2×2 ANOVA was statistically significant (p<0.05), main effects for TNBS, irisin, and TNBS*Ir interaction were recorded as well as all pairwise comparisons. Data are represented as mean±standard deviation.

### EXAMPLE 2

### All Animals Regardless of Treatment Maintained Bodyweight and Normal Eating Behaviours

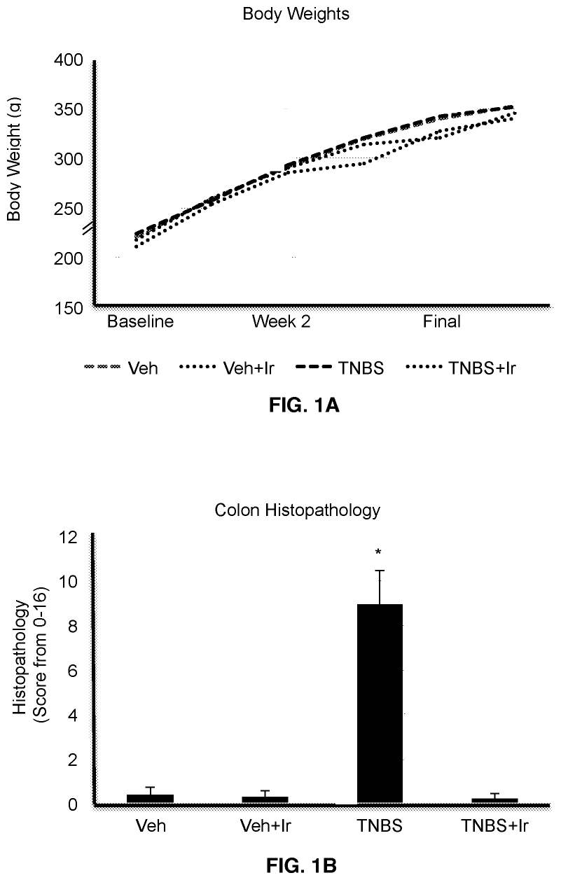

There were no differences in bodyweight (p=0.465) and food intake during the study (FIG. 1A). No animals exhibited overt signs of distress during the entire experimental protocol.

### EXAMPLE 3

### Irisin Treatment During IBD Restored GI Structural Integrity

TNBS animals had a disrupted intestinal epithelial lining, with an associated increase in lamina propria cellularity breaching from the submucosa into the mucosal space as shown by hemotoxylin and eosin staining (FIG. 1B). Associated with this breach was disruption to the crypt structures. TNBS animals also developed mild edema, as well as increased thickness of muscularis externa. Veh+Ir and TNBS+Ir colon histology were comparable to Veh and none showed disruption of the epithelium or elevations in cellularity (FIG. 1B). FIG. 1B shows the microscopic characterization of the colon layers to assess for damage including intestinal cell damage, increased inflammation, and indications of edema (excess fluid). The data shows the TNBS-IBD animals had a significantly elevated overall damage score, whereupon treatment by the present invention, irisin, helped resolve the damage.

### EXAMPLE 4

TNBS resulted in Infiltration of Podoplanin-Positive Structures into the Mucosal Compartment that was Resolved with Irisin Treatment

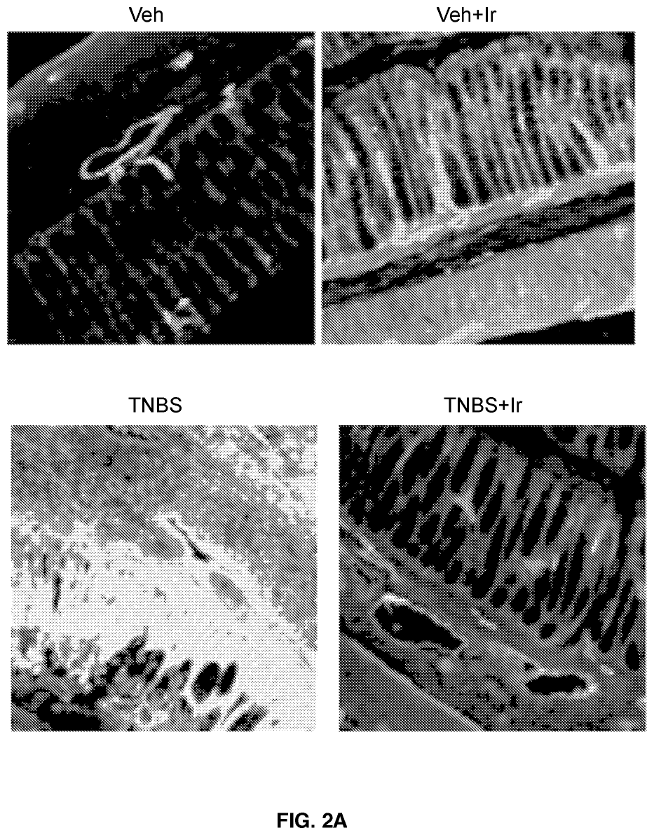

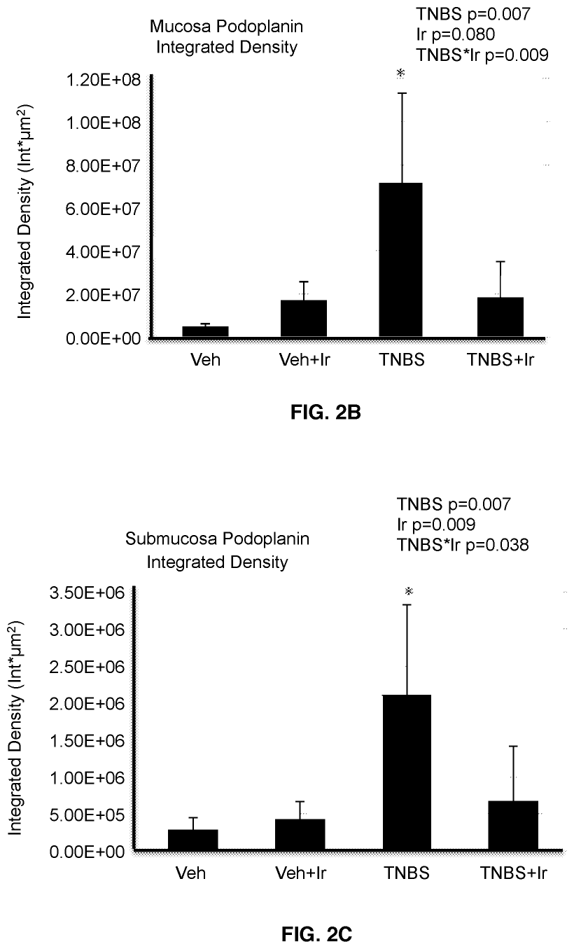

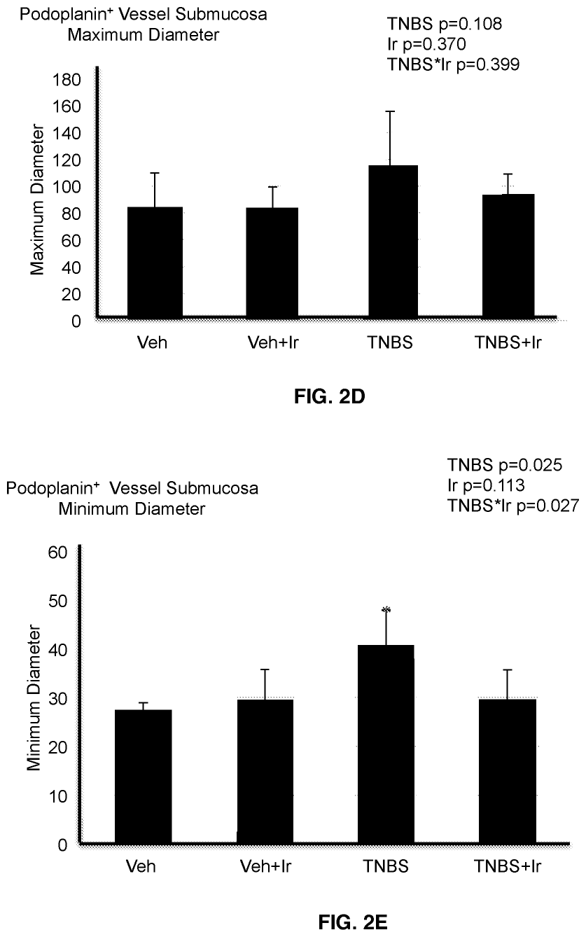

IBD patients have been characterized with elevated density of morphologically abnormal lymphatic vessels in colonic compartments (Geleff 2003, Rahier 2011, D'Alessio 2014). To assess this, colonic sections were characterized via immunofluorescence staining for the canonical lymphatic endothelial marker, podoplanin. In TNBS rats, there was a stark increase in area and expression of podoplanin^{hi} regions compared to Veh animals, evident in both mucosal and submucosal compartments of the colon (FIGS. 2A-2C). These podoplanin^{hi} regions lacked the distinct traversing lymphatic network in the Veh mucosal compartment, and instead became amorphous, unorganized structures in TNBS rodents. This change was quantified in ID, accounting for both the increase in area and in podoplanin expression intensity, noting a mucosal 14 fold ID increase and submucosal 7.5 fold ID increase in TNBS rodents compared to Vehicle (FIGS. 2B-2C). In the TNBS submucosa, podoplanin^{hi} lymphatics were present and had a vascular morphology, with an overall increased vessel diameters (minimum and maximum, FIGS. 2D-2E). The amorphous podoplanin^{hi} regions appeared restricted to the mucosal compartment when present. It is unknown whether these regions are an expansion of the lymphatic capillaries in the lamina propria or a breach/infiltration of lymphatic endothelium below the mucosa into the lamina propria.

To assess for microscopic lymphatic structural changes in the colon, immunofluorescence labelling for a lymphatic marker (podoplanin) was used to identify the lymphatic structures (FIGS. 2B-2D). The structure dictates the function, and in the TNBS-IBD animals, they had an elevated integrated density score, which fits the observations of amorphous, irregular, and large areas of lymphatic structures identified. Integrated density also accounts for protein expression changes, with elevated podoplanin protein expression changes in TNBS-IBD animals supporting inflammation-induced lymphangiogenesis or the formation of new vessels. Treatment with the present invention, irisin, resolved all of these IBD induced changes in the TNBS+Ir animals. TNBS+Ir animals showed a lymphatic architecture comparable to Veh with a remarkable restoration of lymphatic architecture in both mucosal and submucosal compartments (FIG. 2A). Podoplanin-positive ID values for both the mucosa and submucosa were notably lowered in TNBS+Ir animals that accompanied the restoration of typical lymphatic topology in these regions (FIGS. 2B-2C). Although the effects of irisin on lymphatics are unknown, a mild increase was observed in podoplanin ID directly by irisin (FIGS. 2B-2C, at the mucosa effect size=0.191, at the submucosa effect size=0.189). Podoplanin's function is equivocal, but supporting evidence of its functional role include; 1) interaction with galectin-8 in lymphatic endothelium to support their adhesion to the surrounding extracellular matrix, 2) lymphangiogenesis, and 3) lymphocyte trafficking through podoplanin interactions with CCL21 (Cueni 2009, Chen 2016, Kerjaschki 2004). Thus, irisin may be modulating lymphatics by interacting with podoplanin to maintain/restore structural integrity, modulation of lymphangiogenic processes, as well as influencing immune cell chemotaxis based on TNF-α⁺ cellularity changes.

### EXAMPLE 5

The Increased Podoplanin-Positive Density in TNBS is Associated with Elevated TNF-α, with Irisin Ameliorating the Pro-Inflammatory Cytokine Milieu in the Colon

TNBS-induced colitis has been characterized as primarily a Th₁-driven disorder. Comparisons between acute and chronic TNBS-induced colitis support a strong Th₁/Th₁₇-driven response (Alex 2009). How these cytokines are distributed in the local colonic compartment, as well as in correspondence to lymphatic vasculature changes is unknown.

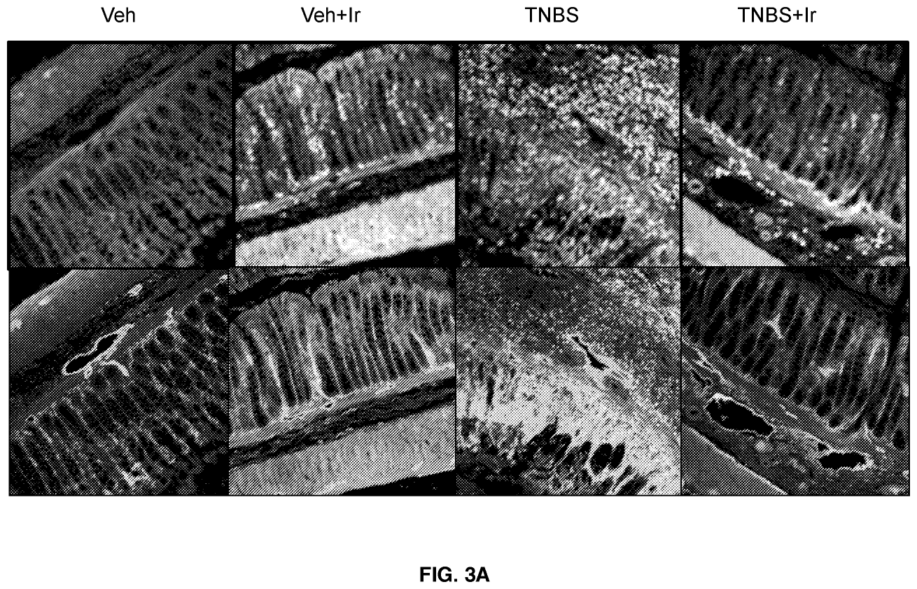

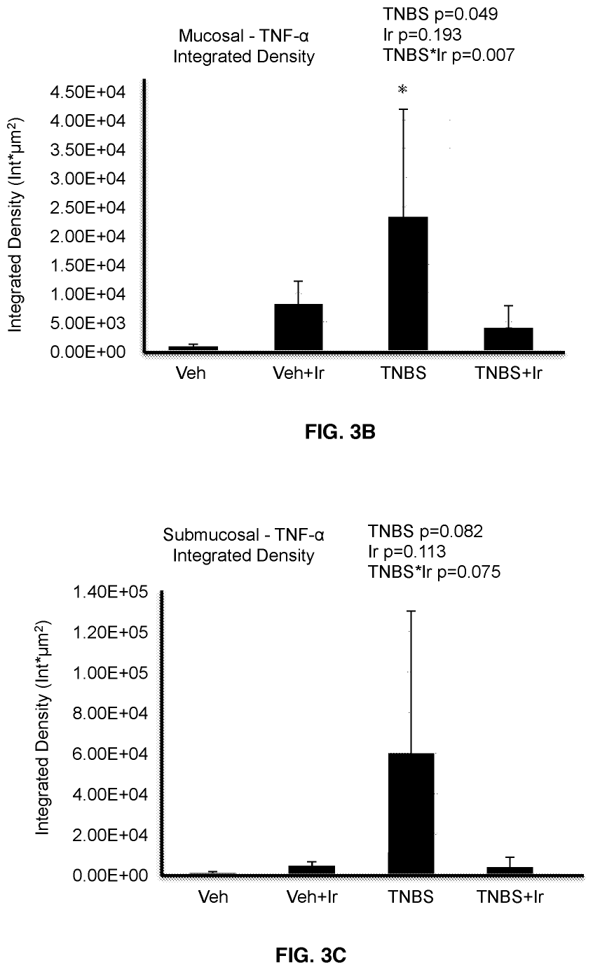

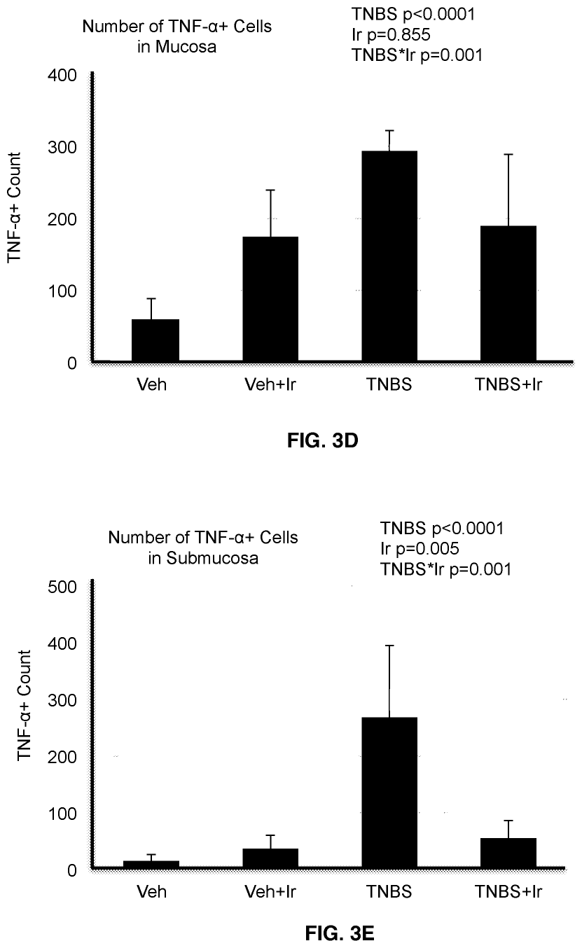

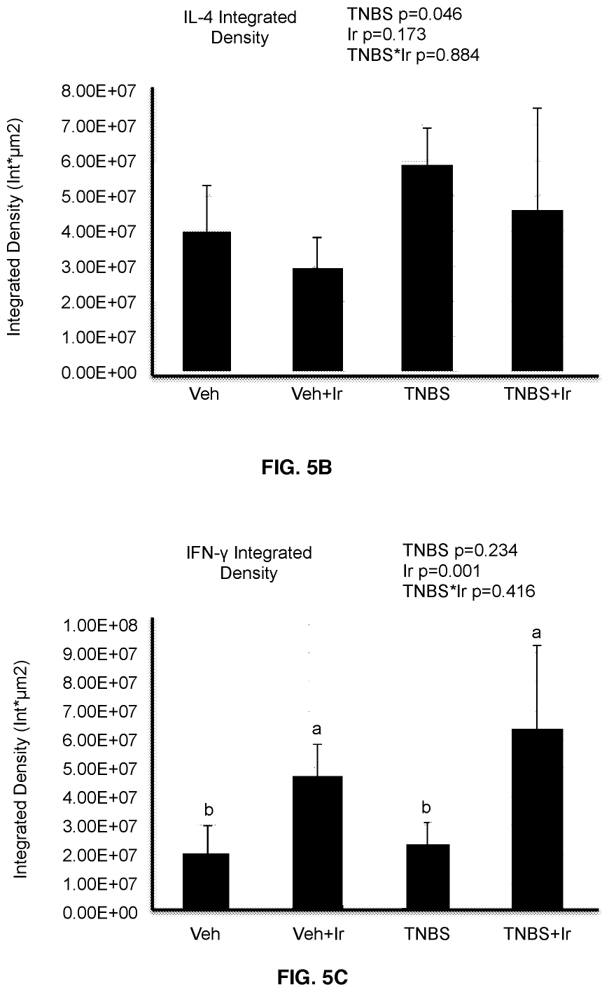

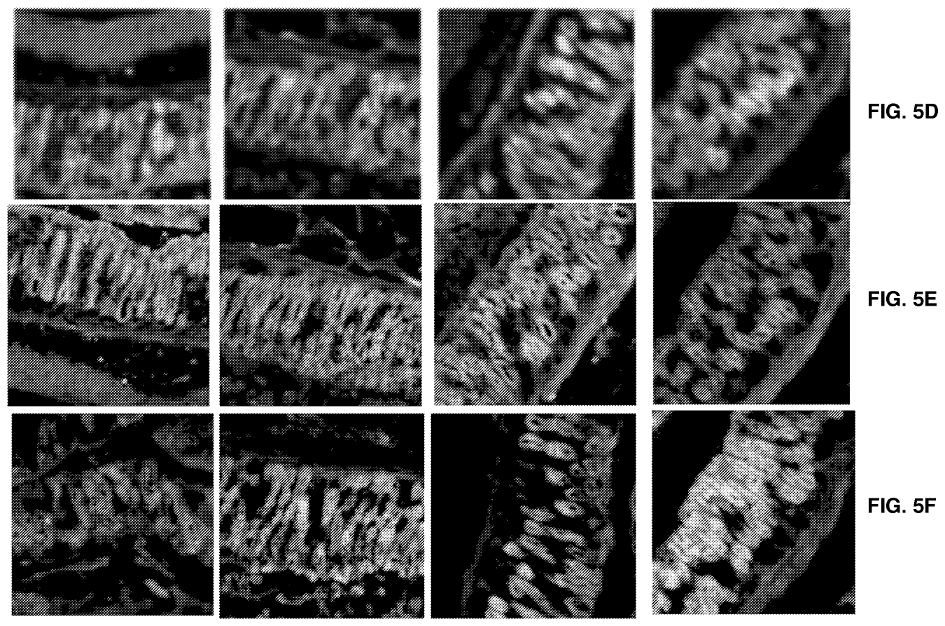

Cytokines involved in inflammatory signaling (including TNF-α, IFN-γ, IL-10, IL-4) were assessed in the colonic compartments in association with lymphatic structural changes (FIGS. 3A-3E, 4A-4C, 5A-5F). To assess for changes in pro-inflammatory processes in the colon, a canonical marker for pro-inflammation, TNF-α, was also tested via immunofluorescence. Notably TNF-α⁺ cells were significantly elevated in TNBS animals, with regards number of cells, area of expression covered per region of interest, and protein expression per cell in both the mucosal and submucosal compartments (FIGS. 3A-3E). Treatment with the present invention, irisin, resolved all of these inflammatory bowel disease induced changes in the TNBS+Ir animals. Vehicle animals had minimal TNF-α expression and cell number, with any present cells localized to the colonic mucosal compartment (FIGS. 3B-3E). TNBS animals had a 6 fold increase in TNF-α⁺ mucosal cell number (effect size=0.550) and 30 fold increase in TNF-α⁺ submucosal cell number (effect size=0.556), with respective 31.9 fold mucosal and 76.2 fold submucosal increases in ID (effect size=0.566, effect size=0.177, respectively) (FIGS. 3D-3E). In the TNBS animals, TNF-α had an elevated integrated density score, supporting increased area of expression as well as increased protein expression (FIGS. 3B-3D). TNF-α has been shown to induce lymphatic proliferation, i.e., lymphangiogenesis, and it is plausible the abnormal TNBS lymphatic morphology is due to this elevation in TNF-α⁺ cell types (Ji 2014). Downregulation of TNF-α may restore the lymphatic architecture, a response observed with irisin treatment after the onset of TNBS. Irisin has been shown to suppress production of Thᵢ cytokines including TNF-α (Shao 2017, Li 2017). Here, TNBS+Ir animals also had significantly lowered TNF-α⁺ ID in both the mucosal (effect size of TNBS*Ir=0.486) and submucosal compartments (effect size of TNBS*Ir=0.495) compared to TNBS alone in association with a restored lymphatic architecture (FIGS. 3B-3C). There was also a reduction in the TNF-α⁺ cell number by irisin treatment. The impact of additional Th₁/Th₂ cytokines, notably IFN-γ, IL-10, and IL-4 (FIGS. 5D-5F) also was characterized. Expression of these cytokines was restricted to the mucosal space. IL-10 expression was elevated in TNBS, with IL-4 also showing an increase in TNBS rats though not statistically elevated. Irisin ameliorated these increases in the TNBS+Ir animals (FIGS. 5A-5B). Interestingly, there was an increase in IFN-y due to irisin treatment (FIG. 5C).

### EXAMPLE 6

### RANKL, a Downstream Target of TNF-α and a Critical Factor for Lymphoid Aggregate Formation, is Elevated in TNBS Animals but Ameliorated by Irisin Treatment

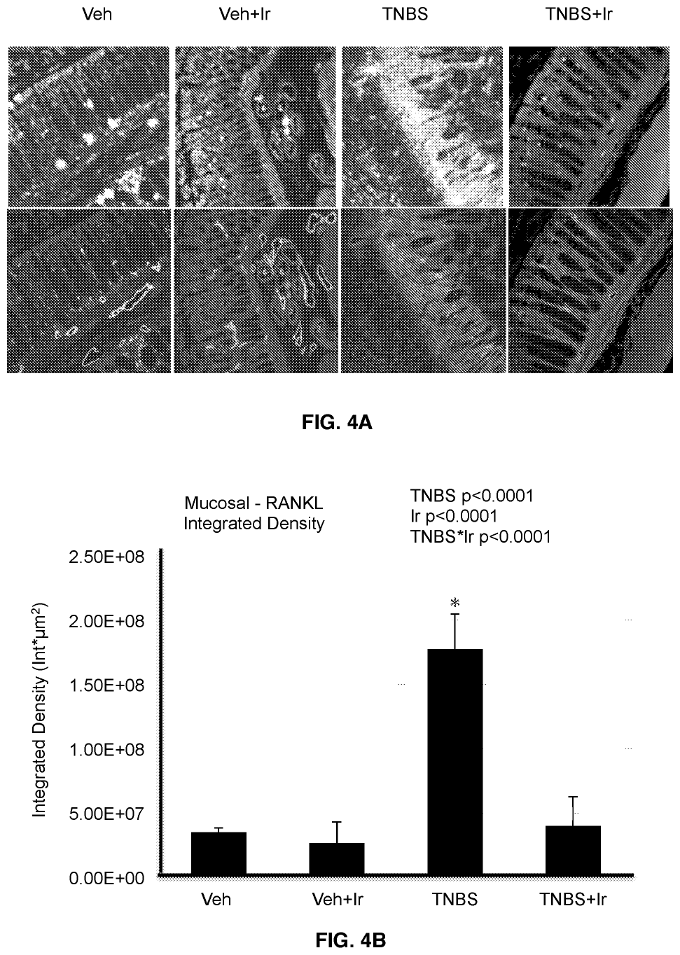

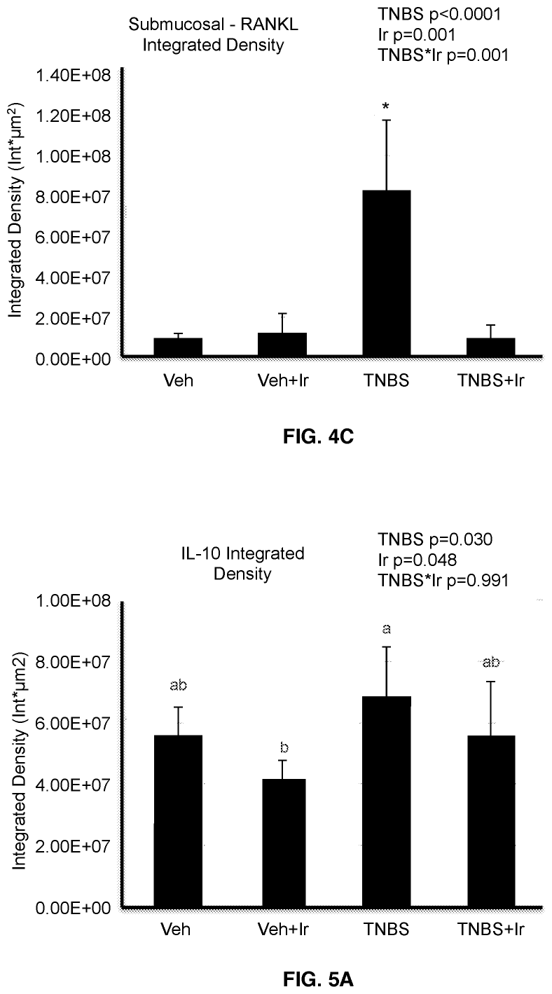

Increased cellularity and lymphoid aggregates in the colon were observed and since RANKL is a known regulator of lymphoid organ formation, RANKL in the colon (FIG. 4A) (Kong, 1999, Aloisi 2006) was quantified. RANKL ID was significantly increased in TNBS animals, in both mucosal and submucosal compartments (effect size=0.864, effect size=0.561, respectively; FIGS. 4B-4C). Significantly, irisin treatment in TNBS+Ir animals ameliorated the elevated RANKL expression to levels comparable to Veh animals (effect size of TNBS*Ir=0.771 for mucosa, effect size=0.595 for submucosa; FIGS. 4B-4C).

### EXAMPLE 7

### Bone Turnover is Altered Due to TNBS Favoring Bone Resorption, While Irisin Treatment Improved Bone Formation

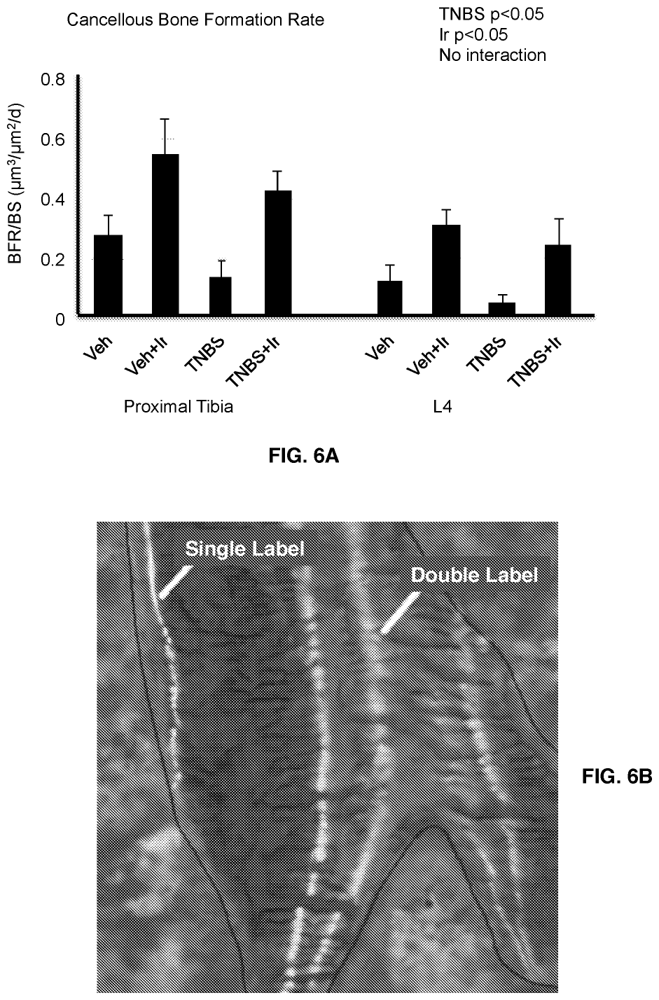

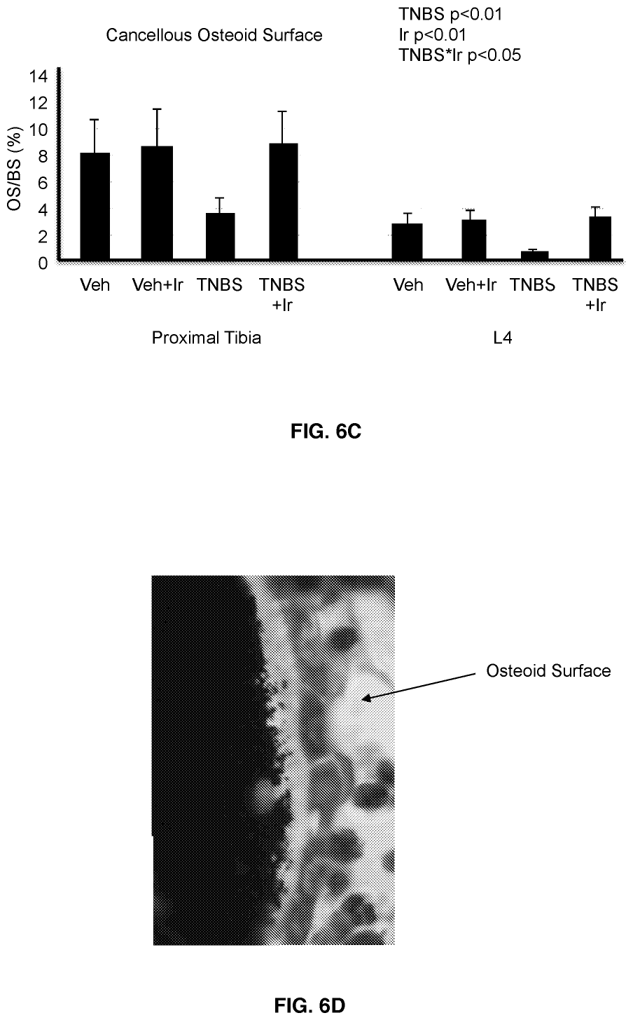

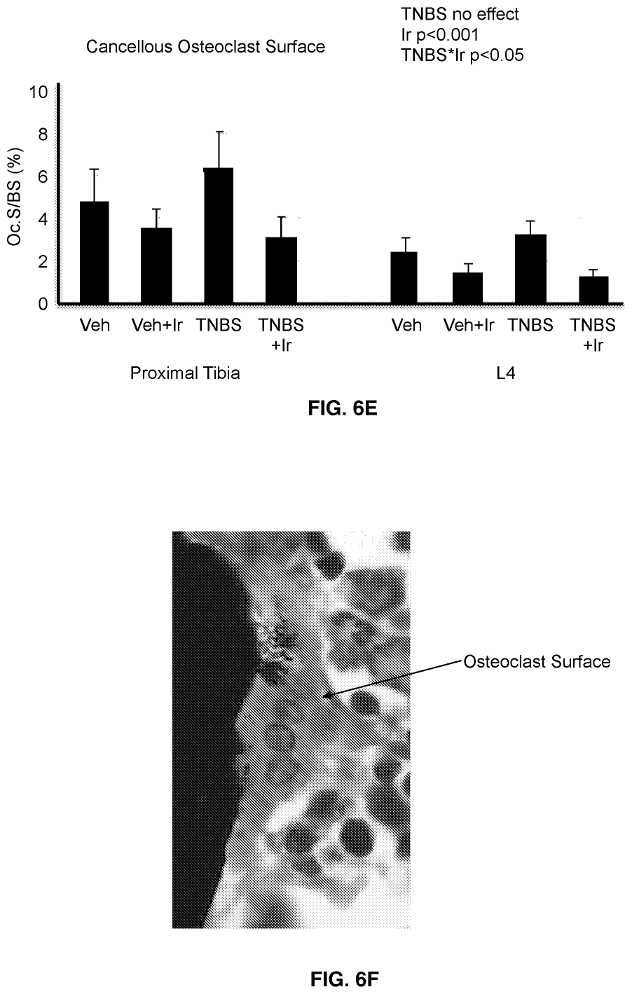

The present invention demonstrated that chronic TNBS resulted in increased osteoclast surface at both the proximal tibia and L4 as well as lower osteoid surface and bone formation rate (FIGS. 6A-6C) (Metzger 2017). Recent data has shown irisin to be a bone anabolic factor with in vivo and cell culture models, demonstrating its impact on increasing osteoblastogenesis and bone formation rate (Zhang 2017, Qiao 2016). The present invention demonstrated that exogenous treatment of irisin resulting in a robust increase in bone formation rate, 1.9-2.4 fold higher than non-irisin treated groups regardless of TNBS (effect size=0.789 for proximal tibia, effect size=0.768; FIG. 6A). Irisin treatment had significant main effects on all measures resulting in a decrease in osteoclast surface and an increase in osteoid surface and bone formation rate at both bone sites (FIGS. 6B-6C). Increased bone formation rate was due to increases in mineralized surface and greater mineral apposition rate indicating both an increase number of osteoblasts and elevated osteoblast activity. This work also demonstrates suppressive effect of irisin on osteoclast surfaces with Veh+Ir and TNBS+Ir had the lowest osteoclast surface regardless of TNBS (effect size of Ir=0.466 in proximal tibia, effect size=0.697 in L4; FIG. 6C). Overall, the irisin treatment completely reversed the directionality of changes due to chronic TNBS treatment on measures of bone turnover. The declines in bone formation rate seen in TNBS were reversed with irisin treatment in both the proximal tibia and 4^{th} lumbar vertebra. Irisin treatment also mitigated the high osteoclast surfaces observed in TNBS rats.

### EXAMPLE 8

In IBD, osteocyte proteins reflect a pro-inflammatory state favoring bone resorption, but irisin treatment alters osteocyte proteins favoring an anabolic state in bone

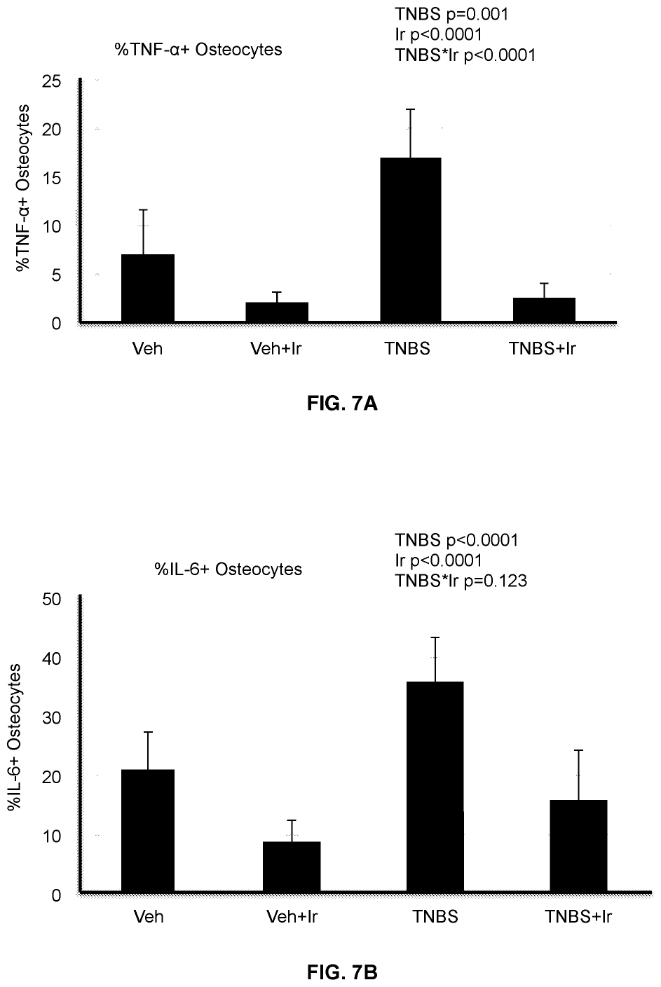

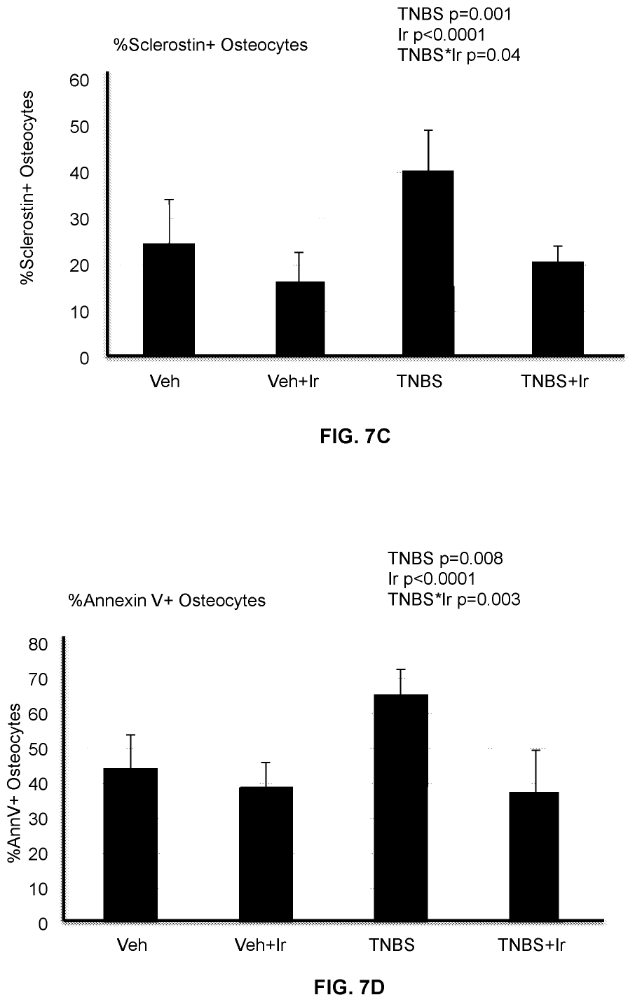

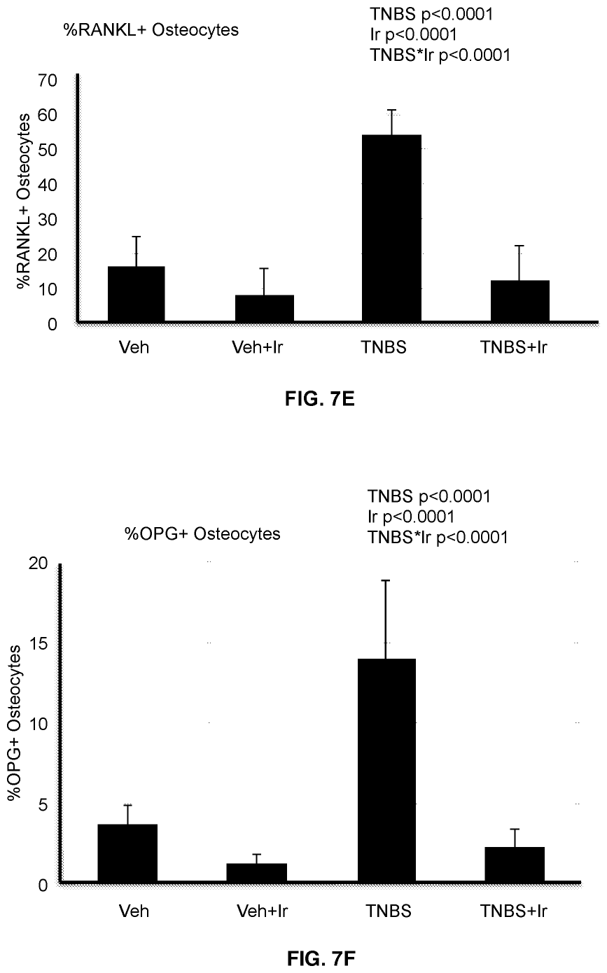

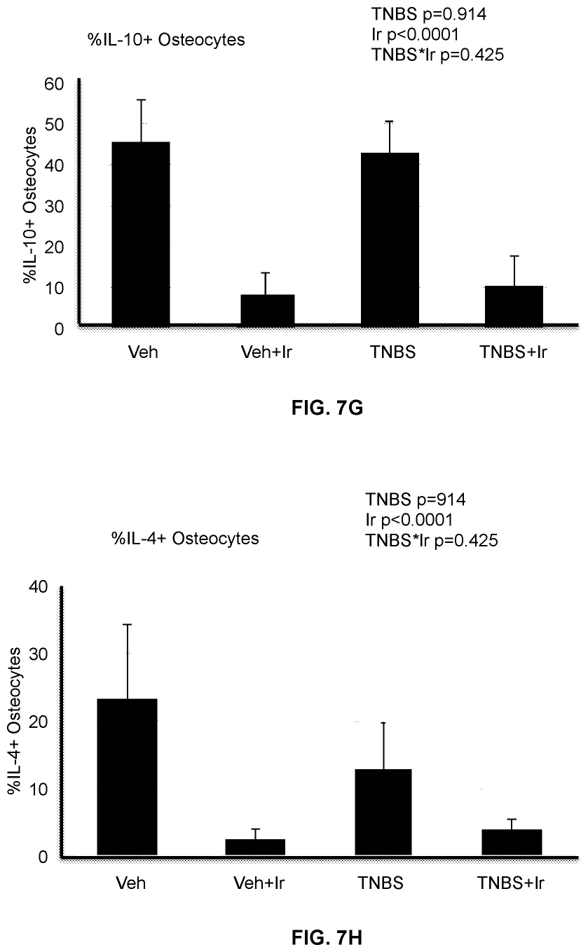

Previously, osteocytes, cells embedded in the bone matrix that release proteins that can impact both osteoblasts and osteoclasts, were examined and the osteocyte protein response showed a pro-inflammatory response in bone due to IBD. (Metzger 2017). TNBS-induced IBD caused an increase in osteocytes positive for TNF-α, IL-6, sclerostin (an inhibitor of bone formation), and osteoclastogenesis regulators, RANKL and OPG (FIGS. 7A-7H). Additionally, osteocyte apoptosis as measured by annexin V was elevated in IBD and was a clear driver of osteoclastic activity (FIG. 7D) (Manolgas 2013). Elevations in all of these factors corresponded with increased bone resorption and decreased bone formation in inflammatory bowel disease. Irisin treatment lowered all of these factors to levels at or lower than Veh, concurrent with significant reductions in osteoclast surface and increased BFR (FIGS. 7A-7H). Osteocyte TNF-α was high in TNBS rats, but lowered down to below that of vehicle treated rats in TNBS+Ir (FIG. 7A). The TNBS-induced elevation in sclerostin, an inhibitor of bone formation rate, was lowered back to the level of vehicle-treated rats in TNBS+Ir (FIG. 7C). Osteoclastogenesis regulator RANKL was high in TNBS, but not different from vehicle-treated in TNBS+Ir (FIG. 7E). Likely the decrease in TNF-α was a major contributor to these changes since sclerostin is upregulated by TNF-α. RANKL and OPG are both influenced by TNF-α, and TNF-α can induce osteocyte apoptosis (Baek 2014, Steeve 2004, Hofbauer 1998, Tan 2006). Other immunological factors like IL-10 and IL-4 were lowered with irisin treatment while TNBS had little impact on IL-10 and TNBS lowered IL-4 (FIGS. 7G-7H). Both IL-4 and IL-10 are proposed to inhibit osteoclast formation; their role in IBD and the impact of their changes with irisin are unknown. These data indicate a Th₁ inflammatory state in bone during inflammatory bowel disease, but this is reversed by irisin treatment.

### EXAMPLE 9

### Methodology for DSS Model

DSS models are analogous to human ulcerative colitis (UC), effecting specifically the large intestine. This animal model version is a very severe and extreme version of ulcerative colitis-induced inflammatory bowel disease in these rats. DSS induction involved dissolving 2% w/v dextran sodium sulfate (DSS) in rodent drinking water for the chronic duration of the study (4-weeks). Rats would develop UC as they drank, with no differences in drinking water intake measured. DSS resulted in more significant sickness and weight loss than the TNBS model. DSS-induced UC-IBD is classically defined to damage the colon, whereas TNBS-induced Crohn's-IBD is classically defined to damage both the colon and small intestine. Both DSS-induced UC and TNBS-induced Crohn's disease are models of IBD, but they model different subclasses of the inflammatory bowel disease. Irisin treatment was the same as in Example 1 with treatment beginning in the second week of DSS administration. Animals were randomly divided into four different groups (n=8/group): Control (Con), Control with irisin (Con+Ir), IBD (DSS), and UC with irisin (DSS+Ir). All animal procedures were approved by the Texas A&M Institutional Animal Use and Care Committee and confirm to the NIH Guide for the Care and Use of Laboratory Animals.

### EXAMPLE 10

### Colon Histopathology DSS Model

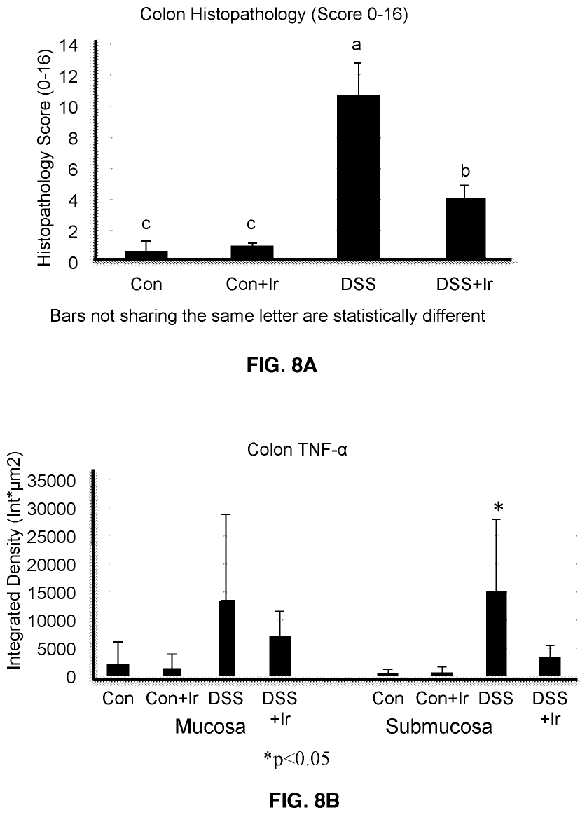

To assess for damage including intestinal cell damage, increased inflammation, and indications of edema (excess fluid), microscopic characterization of the colon layers was done. FIG. 8A shows that the DSS-IBD animals had a significantly elevated overall damage score, whereupon irisin treatment helped resolve a majority of the damage but not entirely to control levels. DSS+Ir animals showed a lack of disruption of the epithelium or elevations in cellularity compared with DSS animals.

### EXAMPLE 11

### Immunofluorescence Labelling of TNF-α Marker in DSS Model

To assess for changes in pro-inflammatory processes in the colon, a canonical marker for pro-inflammation, TNF-α, was quantified via immunofluorescence labelling in the distal colon in the same way as described for the TNBS-IBD study, i.e. Example 1. Integrated density is an aggregate score of area of marker expression and protein expression. In the DSS animals, TNF-α had an elevated integrated density score, supporting increased area of expression as well as increased protein expression due to ulcerative colitis development. Irisin treatment resolved these pro-inflammatory changes, as seen in the DSS+Ir animals having integrated density values comparable to the Control and Con+Ir groups (FIG. 8B). In the DSS animals, TNF-α had an elevated integrated density score, supporting increased area of expression as well as increased protein expression (FIG. 8B). Treatment with the present invention, irisin, in DSS+Ir animals showed a significantly lowered TNF-α⁺ ID in both the mucosal and submucosal compartments compared to DSS animals.

### EXAMPLE 12

### Bone Histomorphometry in DSS Model

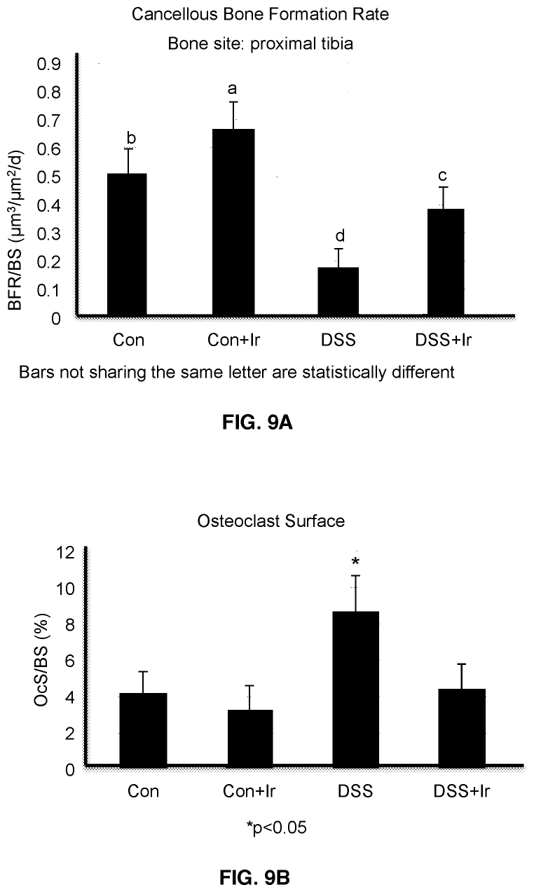

The present invention demonstrated that chronic DSS resulted in increased osteoclast surface and decreased bone formation rate at the proximal tibia. The present invention demonstrated that exogenous treatment of irisin resulting in an increase in bone formation rate compared to the non-irisin treated DSS group (FIG. 9A). Irisin treatment had significant main effects on all measures resulting in a decrease in osteoclast surface and an increase in bone formation rate (FIG. 9B). Increased bone formation rate was due to increases in mineralized surface and greater mineral apposition rate indicating both an increase number of osteoblasts and elevated osteoblast activity. Overall, bone formation rate was decreased in DSS rats while Irisin treatment increased BFR in the DSS+Ir group. Osteoclast surfaces, a marker of bone resorption, were elevated in DSS, but not different from Control in the DSS+Ir group.

### EXAMPLE 13

### In DSS Osteocyte Proteins Reflect a Pro-inflammatory State Favoring Bone Resorption, But Irisin Treatment Alters Osteocyte Proteins Favoring an Anabolic State in Bone

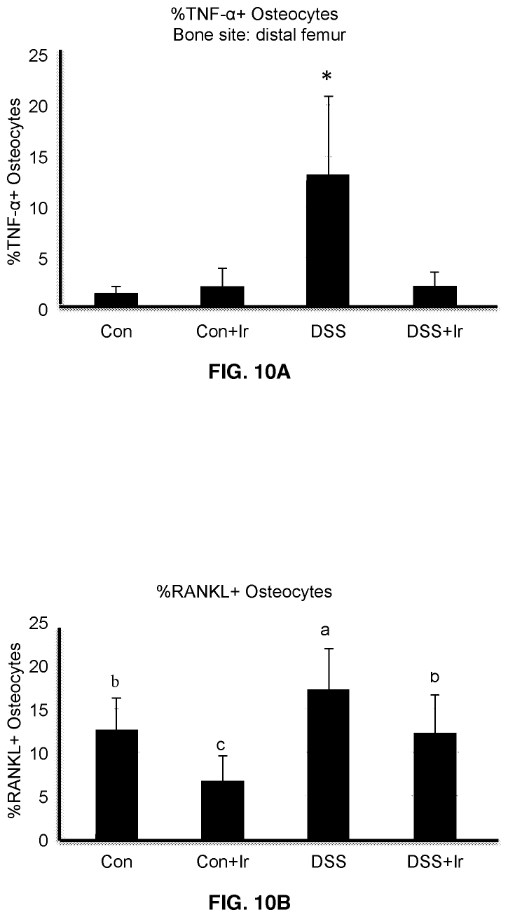

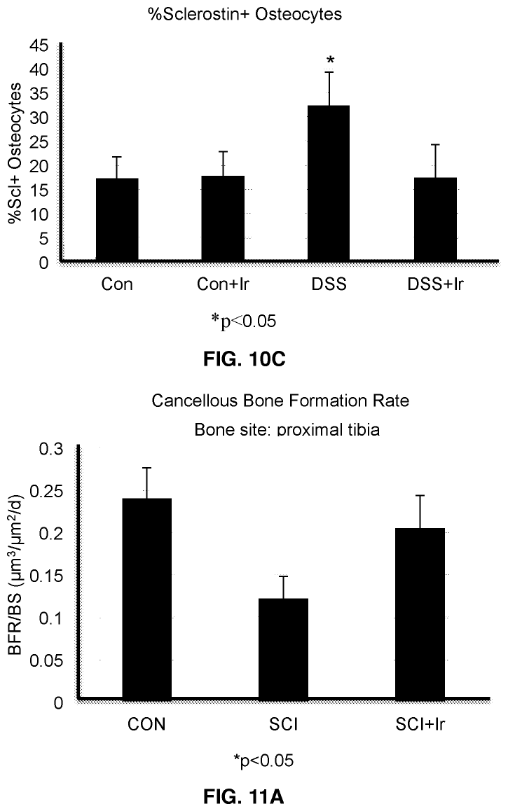

DSS-induced IBD caused an increase in osteocytes positive for TNF-α, sclerostin (an inhibitor of bone formation), and osteoclastogenesis regulator, RANKL (FIGS. 10A-10C). Elevations in all of these factors corresponded with increased bone resorption and decreased bone formation in inflammatory bowel disease. Irisin treatment lowered all of these factors to levels similar to Con, concurrent with significant reductions in osteoclast surface and increased BFR. Osteocyte TNF-α, a pro-inflammatory cytokine, was high in DSS rats, but lowered down to that of control treated rats in DSS+Ir (FIG. 10A). The DSS-induced elevation in sclerostin, an inhibitor of bone formation rate, was comparable to the level of control-treated rats in DSS+Ir (FIG. 10C). Osteoclastogenesis regulator RANKL was high in DSS, was lowered due to irisin treatment in Con and DSS so that DSS+Ir was not different from Con (FIG. 10B).

### EXAMPLE 14

### Irisin Treatment of Spinal Cord Injury

Spinal cord injury is characterized by chronic systemic inflammation in patients which likely contributes to many negative consequences in spinal cord injury including prolonged and drastic bone loss. Treatments for spinal cord injury complications (including immune dysfunction) and SCI-induced bone loss have remained largely refractory to treatments. Therefore, there is a critical need for safe treatments for patients with spinal cord injury. We have demonstrated osteocytes in bone reflected a pro-inflammatory status concurrent with high osteoclast surfaces and low bone formation rate. Animals (2 month old male Sprague Dawley rats) were randomly divided into three different groups (n=8/group): Control (Con), SCI, and SCI with irisin (SCI+Ir). Spinal cord injury animals had a moderate contusion injury at vertebral level T12 resulting in loss of motor function in the lower body. Spinal cord injury rats regained weightbearing on the hindlimbs by day 9 following the injury, but did not fully recovery plantar stepping ability through the 32 days of recovery. Irisin treatment dosing was the same as in Example 1 with twice weekly intraperitoneal injections of irisin for the duration of the experimental period. All animal procedures were approved by the Texas A&M Institutional Animal Use and Care Committee and confirm to the NIH Guide for the Care and Use of Laboratory Animals.

### EXAMPLE 15

### Bone Turnover Is Altered Due to Spinal Cord Injury Favoring Bone Resorption, While Irisin Treatment Improved Bone Formation

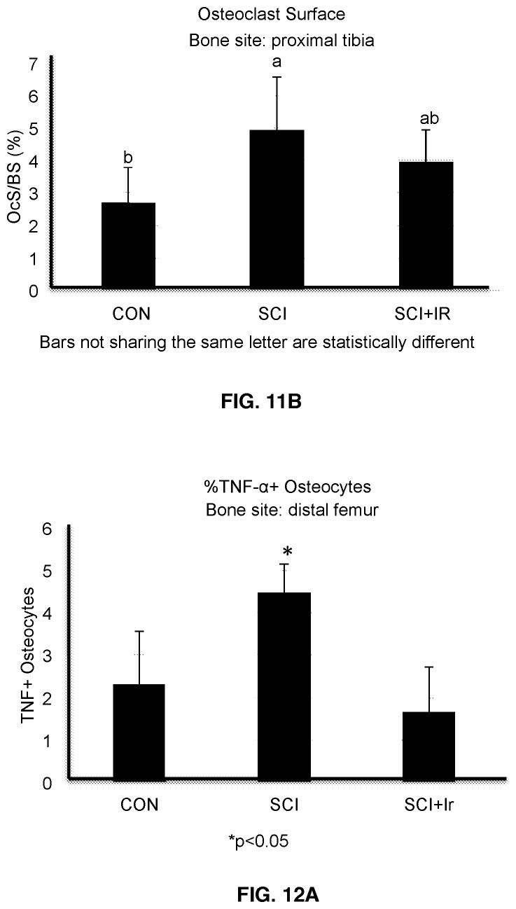

The present invention demonstrated that spinal cord injury resulted in increased osteoclast surface and suppressed bone formation rate at the proximal tibia. The present invention demonstrated that exogenous treatment of irisin resulting in increases in bone formation rate compared to untreated spinal cord injury group. Irisin treatment mitigated the elevated osteoclast surfaces seen in spinal cord injury rats (FIG. 11B).

### EXAMPLE 16

In spinal cord injury, osteocyte proteins reflect a pro-inflammatory state favouring bone resorption, but irisin treatment alters osteocyte proteins favouring an more anabolic state in bone

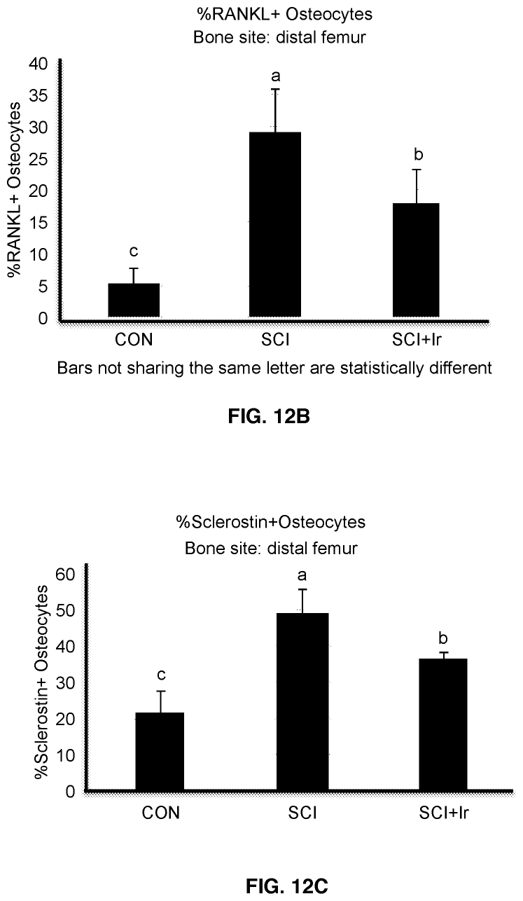

Spinal cord injury caused an increase in osteocytes positive for TNF-α, sclerostin (an inhibitor of bone formation), and osteoclastogenesis regulator, RANKL (FIGS. 12A-12C). Irisin treatment lowered all of these factors compared to the untreated spinal cord injury group. Osteocyte TNF-α, a pro-inflammatory cytokine, was high in spinal cord injury rats, but lowered down to below that of control treated rats in SCI+Ir. The spinal cord injury-induced elevation in sclerostin, an inhibitor of bone formation rate, was also mitigated by Irisin treatment observed in spinal cord injury rats (FIG. 12C). Osteoclastogenesis regulator RANKL was high in spinal cord injury, was lowered due to irisin treatment in SCI+Ir.

### EXAMPLE 17

### Irisin Treatments in a Hindlimb Unloading Model

Hindlimb Unloading (HU) is a ground-based animal model of the weightlessness (i.e. microgravity, or lack of gravity) that astronauts experience in spaceflight. Astronauts experience systemic physiological adaptations to being in space including immune dysregulation, cardiovascular dysfunction, inflammation-induced bone loss, and other spaceflight-induced comorbidities. There is currently a lack of sufficient treatments. We utilized the HU model to test the efficacy of the present invention, irisin, in treatment of microgravity-induced gastrointestinal inflammation and inflammation-induced bone loss. Animals (2 month old male Sprague Dawley rats) were randomly divided into three different groups (n=8/group): Control (Con), HU and HU with irisin (HU+Ir). Irisin treatment was the same as in Example 1. All animal procedures were approved by the Texas A&M Institutional Animal Use and Care Committee and confirm to the NIH Guide for the Care and Use of Laboratory Animals.

### EXAMPLE 18

### Immunofluorescence Labelling of TNF-α Marker in HU Model

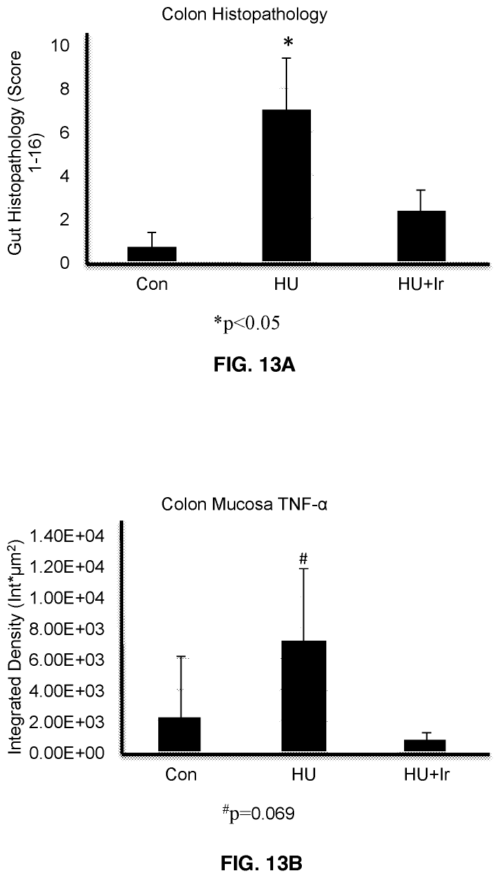

To assess for damage including intestinal cell damage, increased inflammation, and indications of edema (excess fluid) microscopic characterization of the colon layers was done. FIG. 13A shows that show the HU animals had a significantly elevated overall damage score, whereupon irisin treatment with the HU+Ir group helped resolve colonic damage. Further, to assess for changes in pro-inflammatory processes in the colon, a canonical marker for pro-inflammation, TNF-α, was tested via immunofluorescence quantification. In the HU animals, TNF-α had modestly elevated levels of TNF-α expression, supporting simulated weightlessness that astronauts experience may causing inflammation in the digestive system. Irisin treatment resolved these pro-inflammatory changes, as seen in the HU+Ir animals having protein expression values comparable to control groups (FIG. 13B).

### EXAMPLE 19

### Bone Histomorphometry in HU Model

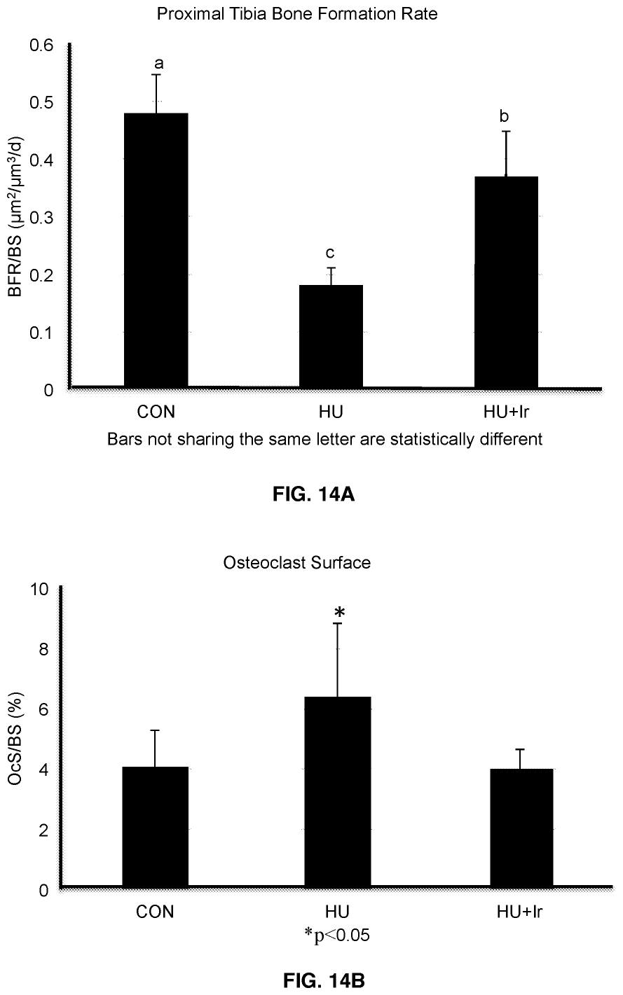

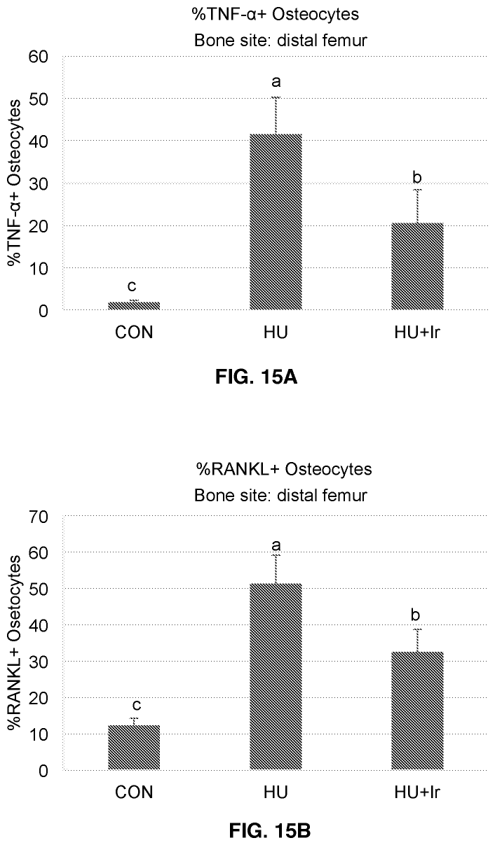

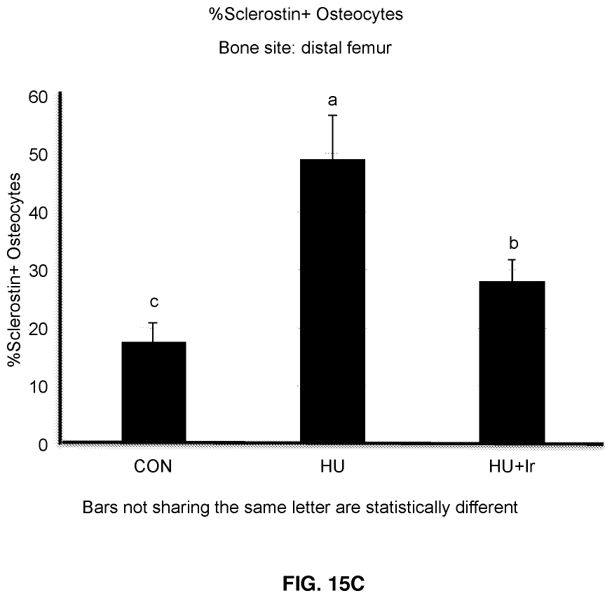

The present invention demonstrated that hindlimb unloading resulted in increased osteoclast surface and suppressed bone formation rate at the proximal tibia. The present invention demonstrated that exogenous treatment of irisin resulting in increases in bone formation rate compared to the untreated HU group. Irisin treatment resolved the high osteoclast surfaces in HU rats with values not different from control levels (FIGS. 14A-14B).

### EXAMPLE 20

### In HU Osteocyte Proteins Reflect a Pro-inflammatory State Favoring Bone Resorption, But Irisin Treatment Alters Osteocyte Proteins Favoring an Anabolic State in Bone

Hindlimb unloading caused an increase in osteocytes positive for TNF-α, sclerostin (an inhibitor of bone formation), and osteoclastogenesis regulator, RANKL (FIGS. 15A-15C). Irisin treatment lowered all of these factors. Osteocyte TNF-α, a pro-inflammatory cytokine, was high in HU rats, but mitigated by treatment with irisin (FIG. 15A). The HU-induced elevation in sclerostin, an inhibitor of bone formation rate, was also mitigated by Irisin treatment observed in HU rats (FIG. 15C). Osteoclastogenesis regulator RANKL was high in HU, was lowered due to irisin treatment in HU+Ir (FIG. 15B).

The following references are cited herein:
Gelef, , Silvana, et al. Oberhuber. Virchows Archly 442.3 (2003): 231-237.
Rahier, et al. Alimentary pharmacology & therapeutics 34.5 (2011): 533-543
D'Alessio S, et al. J Clin Invest 2014; 124:3863-3878.
Cueni, Leah N., et al. Experimental cell research 315.10 (2009): 1715-1723.
Chen, Wei-Sheng, et al. Nature communications 7 (2016).
Kerjaschki, D., et al. Journal of the American Society of Nephrology 15.3 (2004): 603-612.
Alex, Philip, et al. Inflammatory bowel diseases 15.3 (2009): 341-352.
Ji, Hong, et al. Nature communications 5 (2014): 4944.
Shao et al. Biochem Biophysical Res Com, 2017; 10.1016/j.bbrc.2017.04.020.
Li D J, et al. Metabolism, 2017; 68: 31-42.
Metzger C E, et al. J Bone Miner Res,2017; 32(4): 802-813
Zhang J, et al. Bone Research, 2017; 5: 16056.
Qiao X Y, et al. Scientific Reports 2016; 6:18732: DOI: 10.1038/srep18732
Colaianni G, et al, PNAS, 2015; 112(39): 12157-12162.
Manolagas S C, et al. Cell Physiol. 2014; 229: 640-650.
Steeve K T, et al. Cytokine Growth Factor Rev. 2004; 15: 49-60.
Hofbauer et al Biochem Biophys Res Comm. 1998; 250: 776-781.
Tan S D, et at J Dent Res. 2006; 85(10): 2006.

The present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered or modified and all such variations are considered within the scope and spirit of the present invention. The terms in the claims have their plain, ordinary meaning unless otherwise explicitly and clearly defined by the patentee.

1. A method for treating an inflammatory condition in a subject in need of such treatment, comprising the step of:
administering one or more times to the subject a pharmacologically effective dose of irisin or a pharmaceutical composition thereof.

2. The method of claim 1, wherein said dose is about 50 ng/kg of the subject's weight to about 70 ng/kg of the subject's weight.

3. The method of claim 1, wherein said subject is a human, a rodent, or a pig.

4. The method of claim 1, wherein administering irisin to the subject decreases number of TNF-α⁺ cells and expression of TNF-α⁺.

5. The method of claim 1, wherein administering irisin to the subject decreases RANKL, and IL-6 expression.

6. The method of claim 1, wherein administering irisin to the subject increases IFN-γ expression.

7. The method of claim 1, wherein administering irisin to the subject inhibits development of abnormally-structured lymphatic hyperproliferation and reduces overexpression of podoplanin in lymphatic vessels.

8. The method of claim 1, wherein administering irisin to the subject inhibits development of secondary/tertiary lymphoid aggregates therein.

9. The method of claim 1, wherein administering irisin to the subject decreases osteoclast surface and increases osteoid surface and bone formation rate to reduce inflammation-induced alterations in bone turnover.

10. The method of claim 1, wherein administering irisin to the subject decreases a level of at least one osteocyte protein therein.

11. The method of claim 10, wherein the osteocyte protein is TNF-α, IL-6, sclerostin, RANKL, OPG or a combination thereof.

12. The method of claim 1, wherein the inflammatory condition is inflammatory bowel disease, microscopic colitis, Behcet's disease, inflammatory bone loss, primary sclerosing cholangitis, uveitis, rheumatoid/psoriatic arthritis, psoriasis, systemic lupus erythematosus, spinal cord injury, traumatic brain injury, lymphedema, or spaceflight-induced immune dysregulation and associated comorbidities.

13-17. (canceled)

18. A method for treating a spinal cord injury in a subject in need of such treatment, comprising the step of:
administering one or more times to the subject a pharmacologically effective dose of irisin or a pharmaceutical composition thereof.

19. The method of claim 18, wherein the subject is a mammal.

20. The method of claim 19, wherein the mammal is a human, a rodent or a pig.

21. The method of claim 18, wherein said pharmacologically effective dose is from about 50 ng/Kg of the subject's weight to about 70 ng/Kg of the subject's weight.

22. The method of claim 18, wherein administering irisin to the subject decreases osteoclast surface and increases bone formation rate to reduce inflammation-induced alterations in bone turnover.

23. The method of claim 18, wherein administering irisin to the subject decreases level of osteocyte proteins therein.

24. The method of claim 18, wherein the osteocyte protein is TNF-α, sclerostin, or RANKL.

25-32. (canceled)

33. The method of claim 12, wherein the spaceflight-induced immune dysregulation is immune dysregulation and suppression, cardiovascular dysfunction, lymphatic dysfunction, or gastrointestinal dysfunction inflammation-induced bone loss.

34-39. (canceled)
